(19) United States
(12) Patent Application Publication
(10) Pub. No.: US 2024/0309304 A1
(43) Pub. Date: Sep. 19, 2024

(54) DYNAMIC HUMAN ORGAN-ON-CHIP ARRAY FOR HIGH-THROUGHPUT DRUG SCREENING

(71) Applicant: University of North Texas, Denton, TX (US)

(72) Inventors: Yong Yang, Frisco, TX (US); Jiafeng Liu, Denton, TX (US); Kun Man, Denton, TX (US)

(21) Appl. No.: 18/598,039

(22) Filed: Mar. 7, 2024

Related U.S. Application Data
(60) Provisional application No. 63/490,598, filed on Mar. 16, 2023.

(51) Int. Cl. C12M 3/06; C12M 1/00; C12M 1/12; C12M 1/34; C12M 1/42; C12N 5/071; C12N 5/077

(52) U.S. Cl. CPC C12M 23/16; C12M 23/26; C12M 23/58; C12M 25/02; C12M 35/04; C12M 41/46; C12N 5/0656; C12N 5/0688; C12N 2533/90; C12N 2539/00

(57) ABSTRACT

Provided herein is a 3D microfluidic device mimicking an anatomy and physiology of a human lung alveolar interstitium comprising: at least one inlet and one outlet in fluid communication with an inlet chamber and an outlet chamber; an interstitium chamber that comprises an electrospun nanofibrous membrane to support the growth of lung epithelial cells and fibroblasts, at least one inlet chamber and an outlet chamber to provide a growth media to the lung epithelial cells and fibroblasts; a pneumatic chamber separated from the interstitium chamber by a water-impermeable membrane and a source of air in fluid communication with the pneumatic chamber; and an air chamber in fluid communication a second side of the with electrospun nanofibrous membrane of the interstitium chamber, wherein the air chamber is opposite the pneumatic chamber; wherein the integration of inputs and outputs mimics the anatomy and physiology of the human lung alveolar interstitium.

### CROSS-REFERENCE TO RELATED APPLICATIONS

This application claims priority to U.S. Provisional Application Ser. No. 63/490,598, filed Mar. 16, 2023, the entire contents of which are incorporated herein by reference.

### STATEMENT OF FEDERALLY FUNDED RESEARCH

This invention was made with government support under R15GM122953 awarded by the National Institutes of Health. The government has certain rights in the invention.

### TECHNICAL FIELD OF THE INVENTION

The present invention relates in general to the field of organ-on-chip arrays for high-throughput drug screening, and more particularly, to a novel organ-on-a-chip device that mimics human lung alveoli.

### INCORPORATION-BY-REFERENCE OF MATERIALS FILED ON COMPACT DISC

Not applicable.

### BACKGROUND OF THE INVENTION

Without limiting the scope of the invention, its background is described in connection with in vitro models of organs.

Pathophysiologically relevant in vitro models are critical to understanding and treating human disease. Current preclinical models, such as animal models, conventional 2D models, and 3D human organoids, have limitations. Animal studies exhibit different symptoms from humans that often lead to inaccurate results in drug testing. 2D models, in which human primary cells or cell lines are cultured on flat plastic surfaces, behave differently from their in vivo counterparts and fail to replicate the in vivo phenotypes or functions of native tissues. 3D models, such as organoids, fail to model in vivo microenvironments, such as the air-liquid interface (ALI) of human lungs.

Moreover, all these models lack physiologically relevant mechanical characteristics, such as interstitial flow and/or lung breathing movement for human lungs. Organ-on-a-chip systems have recently attracted great attention because they can recapitulate key anatomical and pathophysiological characteristics and imitate the primary functions of human organs. Cell culture and analysis processes are also easier to perform on organ chips than on animal models. Several human lung fibrosis chips have also been developed to replicate stromal-vascular and stromal-epithelial interfaces.

Despite these advances, what is needed are novel 3D in vitro models that recapitulate key anatomical and pathophysiological characteristics and imitate the primary functions of the human lung.

### SUMMARY OF THE INVENTION

As embodied and broadly described herein, an aspect of the present disclosure relates to a 3D microfluidic device mimicking an anatomy and physiology of a human lung alveolar interstitium comprising: a housing defining a cavity, at least one inlet and one outlet, wherein the inlet and outlet are in fluid communication with an inlet chamber and an outlet chamber; an interstitium chamber positioned in the cavity that comprises an electrospun nanofibrous membrane that supports the growth of lung epithelial cells and fibroblasts, wherein the electrospun nanofibrous membrane is in fluid communication with the at least one inlet chamber and an outlet chamber to provide a growth media to the lung epithelial cells and fibroblasts; a pneumatic chamber separated from the interstitium chamber by a water-impermeable membrane and a source of air in fluid communication with the pneumatic chamber; and an air chamber in fluid communication a second side of the with electrospun nanofibrous membrane of the interstitium chamber, wherein the air chamber is opposite the pneumatic chamber; wherein the integration of inputs and outputs mimics the anatomy and physiology of the human lung alveolar interstitium. In one aspect, the inlet aperture and the outlet aperture allow for injection of at least one of fluid, gas and solid material within and through the device. In another aspect, the device is configured to be filled with cellular components on the electrospun nanofibrous membrane. In another aspect, the microfluidic device further comprises one or more seals that separate the interstitium chamber, pneumatic chamber, and air chamber. In another aspect, the microfluidic device further comprises a pump to provide pneumatic pressure in the pneumatic chamber. In another aspect, the device comprises a material selected from a group consisting of glass, silicon, polysiloxane, polydimethylsiloxane, and optically transparent polymers. In another aspect, the electrospun nanofibrous membrane has at least one of: physiological interstitial matrix stiffness or physiological 3D breathing mechanical stretch. In another aspect, the interstitium chamber further comprises a collagen I-fibrin blend gel. In another aspect, the interstitium chamber comprises one or more lung cell lines, lung primary cell cultures, alveolar epithelial cells, fibroblasts, immune cells or combinations thereof.

As embodied and broadly described herein, an aspect of the present disclosure relates to a lab-on-a-chip comprising a 3D microfluidic device mimicking an anatomy and physiology of a human lung alveolar interstitium comprising: a housing defining a cavity, at least one inlet and one outlet, wherein the inlet and outlet are in fluid communication with an inlet chamber and an outlet chamber; an interstitium chamber positioned in the cavity that comprises an electrospun nanofibrous membrane that supports the growth of lung epithelial cells and fibroblasts, wherein the electrospun nanofibrous membrane is in fluid communication with the at least one inlet chamber and an outlet chamber to provide a growth media to the lung epithelial cells and fibroblasts; a pneumatic chamber separated from the interstitium chamber by a water-impermeable membrane and a source of air in fluid communication with the pneumatic chamber; and an air chamber in fluid communication a second side of the with electrospun nanofibrous membrane of the interstitium chamber, wherein the air chamber is opposite the pneumatic chamber; wherein the integration of inputs and outputs mimics the anatomy and physiology of the human lung alveolar interstitium. In one aspect, the inlet aperture and the outlet aperture allow for injection of at least one of fluid, gas and solid material within and through the device. In another aspect, the device is configured to be filled with cellular components on the electrospun nanofibrous membrane. In another aspect, the lab-on-a-chip further comprises one or more seals that separate the interstitium chamber, pneumatic chamber, and air chamber. In another aspect, the lab-on-a-chip further comprises a pump to provide pneumatic pressure in the pneumatic chamber. In another aspect, the device comprises a material selected from a group consisting of glass, silicon, polysiloxane, polydimethylsiloxane, and optically transparent polymers. In another aspect, the electrospun nanofibrous membrane has at least one of: physiological interstitial matrix stiffness or physiological 3D breathing mechanical stretch. In another aspect, the interstitium chamber further comprises a collagen I-fibrin blend gel. In another aspect, the interstitium chamber comprises one or more lung cell lines, lung primary cell cultures, alveolar epithelial cells, fibroblasts, or combinations thereof.

As embodied and broadly described herein, an aspect of the present disclosure relates to a kit comprising a lab-on-a-chip comprising: a 3D microfluidic device mimicking an anatomy and physiology of a human lung alveolar interstitium comprising: a housing defining a cavity, at least one inlet and one outlet, wherein the inlet and outlet are in fluid communication with an inlet chamber and an outlet chamber; an interstitium chamber positioned in the cavity that comprises an electrospun nanofibrous membrane that supports the growth of lung epithelial cells and fibroblasts, wherein the electrospun nanofibrous membrane is in fluid communication with the at least one inlet chamber and an outlet chamber to provide a growth media to the lung epithelial cells and fibroblasts; a pneumatic chamber separated from the interstitium chamber by a water-impermeable membrane and a source of air in fluid communication with the pneumatic chamber; and an air chamber in fluid communication a second side of the with electrospun nanofibrous membrane of the interstitium chamber, wherein the air chamber is opposite the pneumatic chamber; wherein the integration of inputs and outputs mimics the anatomy and physiology of the human lung alveolar interstitium.

As embodied and broadly described herein, an aspect of the present disclosure relates to a method mimicking an anatomy and physiology of a human lung alveolar interstitium with a lab-on-a-chip comprising a 3D microfluidic device comprising: providing a housing defining a cavity, at least one inlet and one outlet, wherein the inlet and outlet are in fluid communication with an inlet chamber and an outlet chamber; inserting lung epithelial cells and fibroblasts into an interstitium chamber positioned in the cavity that comprises an electrospun nanofibrous membrane that supports the growth of the lung epithelial cells and fibroblasts, wherein the electrospun nanofibrous membrane is in fluid communication with the at least one inlet chamber and an outlet chamber to provide a growth media to the lung epithelial cells and fibroblasts; applying pneumatic pressure to the device into a pneumatic chamber separated from the interstitium chamber by a water-impermeable membrane and a source of air in fluid communication with the pneumatic chamber; and providing an air chamber in fluid communication a second side of the with electrospun nanofibrous membrane of the interstitium chamber, wherein the air chamber is opposite the pneumatic chamber; wherein the integration of inputs and outputs mimics the anatomy and physiology of the human lung alveolar interstitium. In one aspect, the inlet aperture and the outlet aperture allow for injection of at least one of fluid, gas and solid material within and through the device. In another aspect, the device is configured to be filled with cellular components in the electrospun nanofibrous membrane. In another aspect, the method further comprises one or more seals that separate the interstitium chamber, pneumatic chamber, and air chamber. In another aspect, the method further comprises a pump to provide pneumatic pressure in the pneumatic chamber. In another aspect, the device comprises a material selected from a group consisting of glass, silicon, polysiloxane, polydimethylsiloxane, and optically transparent polymers. In another aspect, the electrospun nanofibrous membrane has at least one of: physiological interstitial matrix stiffness or physiological 3D breathing mechanical stretch. In another aspect, the interstitium chamber further comprises a collagen I-fibrin blend gel. In another aspect, the interstitium chamber comprises one or more lung cell lines, lung primary cell cultures, alveolar epithelial cells, fibroblasts, or combinations thereof.

As embodied and broadly described herein, an aspect of the present disclosure relates to a high-throughput method of determining the effectiveness of a candidate drug that impacts lung alveoli, the method comprising: providing a housing defining an array of test wells that each comprise a first and a second cavity, each of the cavities comprising at least one inlet and one outlet, wherein the inlet and outlet are in fluid communication with an inlet chamber and an outlet chamber; inserting lung epithelial cells and fibroblasts into an interstitium chamber positioned in the housing that comprises an electrospun nanofibrous membrane that supports the growth of the lung epithelial cells and fibroblasts, wherein the electrospun nanofibrous membrane is in fluid communication with the at least one inlet chamber and an outlet chamber to provide a growth media to the lung epithelial cells and fibroblasts; applying pneumatic pressure into a pneumatic chamber separated from the interstitium chamber by a water-impermeable membrane and a source of air in fluid communication with the pneumatic chamber; and providing an air chamber in fluid communication a second side of the electrospun nanofibrous membrane of the interstitium chamber, wherein the air chamber is opposite the pneumatic chamber; wherein the integration of inputs and outputs mimics the anatomy and physiology of the human lung alveolar interstitium; administering a candidate drug to at least a first test well, and a placebo to at least a placebo test well; and determining if the candidate drug modifies one or more parameters associated with lung function in the at least first test well when compared to the placebo test well over a course of treatment with the candidate drug.

### BRIEF DESCRIPTION OF THE DRAWINGS

For a more complete understanding of the features and advantages of the present invention, reference is now made to the detailed description of the invention along with the accompanying figures and in which:

FIGS. 1A to 1D shows the Design and fabrication of human lung alveolar interstitium chip. (FIG. 1A) Schematic illustration of human lung alveolus. (FIG. 1B) Design, (FIG. 1C) photo, and (FIG. 1D) fabrication of the chip. The red box in (FIG. 1D) is the cross-sectional view of A-A of the interstitium chamber fabrication.

FIGS. 2A to 2C shows the characterization of electrospun nanofibrous membranes. (FIG. 2A) Scanning electron micrographs (SEM) of nanofibrous membranes of small (S), medium (M), and large (L) pore sizes. Scale bars: 20 μm. (FIG. 2B) Pore size distributions of these nanofibrous membranes. The insets magnify the main pore size distributions. (FIG. 2C) Fiber diameter distributions of these nanofibrous membranes.

FIGS. 3A to 3D show that nanofibrous membrane promoted the formation of tight epithelium. (FIG. 3A) Representative immunofluorescence images of A549 cells cultured on the PCL flat control and nanofibrous membranes (SEM images are presented in the insets) for 10 days. Green: ZO-1; blue: nuclei. (FIG. 3B) Western blotting of ZO-1, ZO-3, occludin, and E-cadherin of A549 cells cultured on the flat control and nanofibrous membranes with small (S), medium (M), and large (L) pore sizes (n=3). The data were normalized to the mean value of the flat control. (FIG. 3C) Photos of transwell inserts attached with the nanofibrous membrane. (FIG. 3D) Permeability of 4 kDa dextran across the epithelial cell layer on the nanofibrous membranes. *:p<0.05 compared to the flat control. #:p<0.05 between groups.

FIGS. 4A to 4C show that the interstitium chip promoted epithelial barrier function. Low and high magnification SEM images of the epithelial monolayer (FIG. 4A) on the chip and (FIG. 4B) in the transwell model. (FIG. 4C) Comparison of dextran permeability on the chip and the transwell model after 1-week of culture. *:p<0.05 compared to transwell model.

FIGS. 5A to 5D show the long term culture of the lung interstitium chip. (FIG. 5A) Low and (FIG. 5B) high magnification SEM images of the epithelium on the chip. The yellow arrows in (FIG. 5B) point to the apical microvilli. (FIG. 5C) SEM image of the layered structure of the epithelium on the chip. (FIG. 5D) Dextran permeability of the epithelium after 4-week culture in the chip. *:p<0.05 compared to the transwell models.

FIGS. 6A and 6B show a toxicity assessment of MWCNTs on the chip and transwell model. (FIG. 6A) Percentage of MWCNTs penetrated across the epithelium. (FIG. 6B) Expression of IL-8 of the fibroblasts after the epithelium exposed to MWCNTs for 24 hours, *:p<0.05 compared to the transwell model.

FIGS. 7A to 7F show the engineering of the multi-chip array. (FIG. 7A) Assembly of air chamber inserts and interstitium chambers. The parts of the multi-chip array (FIG. 7B) are assembled into the array (FIG. 7C). The black box shows the pin that assists the alignment and assembly of interstitium layer and pneumatic layers. (FIG. 7D) A single chip design. (FIG. 7E) Photo of a multi-chip array. (FIG. 7F) SolidWork simulation indicates the interstitium shear stress in a physiological range.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

The present invention is a human lung alveolar interstitium chip that was developed to imitate key alveolar microenvironmental factors including: an electrospun nanofibrous membrane as the analogue of the basement membrane for co-culture of epithelial cells with fibroblasts embedded in 3D collagenous gels and immune cells; physiologically relevant interstitial matrix stiffness; interstitial fluid flow; and 3D breathing-like mechanical stretch. The biomimetic chip substantially improved epithelial barrier function compared to transwell models. Moreover, the chip having a gel made of a collagen I-fibrin blend as the interstitial matrix sustained the interstitium integrity and further enhanced the epithelial barrier resulting in a longevity that extended beyond eight weeks. The assessment of multiwalled carbon nanotube toxicity on the chip was in line with the animal study.

Being responsible for the exchange of oxygen and carbon dioxide, the alveoli, tiny air sacs in human lungs, also serve as the entry gateway for airborne particles such as engineered nanomaterials, air pollutants, and pathogens to enter the human body. Alveoli also provide a route for pulmonary drug delivery. As an example, engineered nanomaterials, especially when in their aerosolized form, can be inhaled into lungs potentially causing significant public health concerns [1]. It has been shown that inhaled carbon nanotubes (CNTs), a major class of engineered nanomaterials [2], cross the alveolar epithelial barrier and enter the interstitium, inducing progressive interstitial lung fibrosis in mice in weeks [3]. In addition to negative health implications, researchers seek to leverage the large alveolar surface area for inhalation-based delivery of drug-loaded nanoparticles, a cutting-edge technology for lung cancer therapy [4]. Thus, pathophysiologically relevant preclinical alveolus models are critical for understanding and treatment of lung diseases and toxicity assessment of engineered nanomaterials.

Current preclinical models (e.g., animal models, conventional 2D models, and lung organoids) have limitations. Differences in the anatomy, physiology, and genomics between animals and humans make conventional animal models inconsistent and inaccurate, limiting their translation to clinical studies [5]. In 2D models, human primary cells or cell lines are cultured on flat plastic surfaces, which leads to different behaviors compared to their in vivo counterparts that interferes with the replication of in vivo phenotypes or functions observed in native tissues [6]. Lung organoids replicate complex 3D structures and functions of the lung but cannot model some key in vivo lung microenvironmental features like the air-liquid interface (ALI) [7, 8]. Moreover, all these models lack physiologically relevant mechanical characteristics, such as interstitial flow and/or lung breathing movement.

Organ-on-a-chip systems can replicate organismal-level function and have recently attracted great attention [9, 10]. The human lung chips model the ALI and alveolus-capillary interface, mimic breath movement [9], and have been adapted to model several diseases and to test therapeutic drugs [11, 12]. Several human lung fibrosis chips have also been developed to replicate stromal-vascular and stromal-epithelial interfaces [13, 14]. However, these chips do not mimic the basement membrane, interstitium stiffness, or breathing movement critical for lung functions and disease [15, 16]. A study by the present inventors reveals that mechanical stretch dimensionality (i.e., 3D stretch similar to native alveoli) is critical to epithelium formation [17]. Due to the missing key alveolar microenvironmental features, the epithelial barrier function is not sustained, and most of these chips are only viable for 1-3 weeks [9, 11, 18, 19]; therefore, they are not suitable for investigation of chronic diseases.

However, the present inventors recognized that these chips do not mimic the basement membrane or breathing movement critical for lung functions and cancer; elevated stiffness and stretch (especially those similar to fibrotic conditions) contribute significantly to causing cancer phenotypes while promoting aggressive behavior in a fashion not replicated by conventional static conditions. Drug screening conducted under static conditions can result in false leads while missing potential therapeutic candidates. Furthermore, the evaluation of therapeutic candidates demands high-throughput screen platforms of pathophysiological relevance.

The present invention is a human alveolar interstitium chip that uses an electrospun nanofibrous membrane for co-culture of alveolar epithelial cells with lung fibroblasts encapsulated in 3D collagenous hydrogels. The chip also provides interstitial flow and 3D mechanical stretch of physiological relevance to the cells. The pore size of the nanofibrous membrane was optimized to promote epithelium formation. The interstitium chip exhibited enhanced epithelial barrier function with longevity extended beyond eight weeks. Furthermore, the inventors assessed the penetration of multi-walled CNTs (MWCNTs) across the epithelium of the chips, which showed results consistent with the in vivo study [20].

As used herein, the term “substrate” refers to a variety of different biocompatible materials including polymeric materials, agent-binding surfaces, cell-binding surfaces, plastics, glass, or other manufacturable materials such as ceramic, metal, resin, gel, glass, silicon, glass-ceramics, and composites thereof. The substrate can be functionalized to, e.g., add a charge to the surface of the substrate to increase the adhesion of cells.

For cell and/or fluid delivery to and from a chamber or channel, the cell and/or fluid can be delivered through a top or side opening, a via, a tube, or other suitable structure for transporting cells and/or fluids. The via or tube will be made from constructed from a biocompatible material, such as a polymer, plastic, glass, or other biocompatible material. Generally, each chamber will be in fluidic communication through a via or channel. Other types of fluidic connectors and/or reservoirs suitable for bringing fluidic communication (e.g., recreating blood flow through specific organs by incorporating dynamic flow, to mimic a pneumatic pressure, to mimic oxygen pressure) between chambers and/or the environment outside the device will be readily apparent to one of ordinary skill in the art.

Non-limiting examples of materials for use as a substrate with the present invention include, but are not limited to, polymeric materials such as polydimethylsiloxane (PDMS), polyurethane-methacrylate (PUMA), polymethylmethacrylate (PMMA), polyethylene, polyester (PET), polytetrafluoroethylene (PTFE), polycarbonate, parylene, polyvinyl chloride, fluoroethylpropylene, lexan, polystyrene, cyclic olefin copolymers, polyurethane, polyestercarbonate, polypropylene, polybutylene, polyacrylate, polycaprolactone, polyketone, polyphthalamide, polyacrylonitrile, polysulfone, epoxy polymers, thermoplastics, fluoropolymer, and polyvinylidene fluoride, polyamide, polyimide), cellulose acetate, fused silica, ceramic, glass (organic), metals, inorganic materials (glass, quartz, silicon, GaAs, silicon nitride), and/or other materials and combinations thereof.

The chambers and the microchannels connecting the same can be surface-modified chemically to enhance wetting or to assist in the adsorption of the tissues or cells. Non-limiting examples of surface-modifying chemicals can include, but are not limited to, silanes such as trimethylchlorosilane (TMCS), hexamethyldisilazane (HMDS), (Tridecafluoro-1,1,2,2-tetrahydrooctyl)trichlorosilane, chlorodimethyloctylsilane, Octadecyltrichlorosilane (OTS) or γ-methyacryloxypropyltrimethyoxy-silane; polymers such as acrylic acid, acrylamide, dimethylacrylamide (DMA), 2-hydroxyethyl acrylate, polyvinylalcohol (PVA), poly(vinylpyrrolidone (PVP), poly(ethylene imine) (PEI), Polyethylene glycol (PEG), epoxy poly(dimethylacrylamide (EPDMA), PEG-monomethoxyl acrylate; surfactants such as Pluronic surfactants, Poly(ethylene glycol)-based (PEG) surfactants, sodium dodecylsulfate (SDS) dodecyltrimethylammonium chloride (DTAC), cetyltriethylammonium bromide (CTAB), or Polybrene (PB); cellulose derivatives such as hydroxypropylcellulose (HPC), or hydroxypropylmethylcellulose (HPMC); amines such as ethylamine, diethylamine, triethylamine, or triethanolamine, fluorine-containing compounds such as those containing polytetrafluoroethylene (PTFE) or TEFLON®.

Adhesion-promoting materials for use with the present invention can include but are not limited to: poly-L-Lysine, poly-D-Lysine, high-molecular-weight cationic copolymer of polyacrylamide and quaternized cationic monomers, poly dopamine, collagen, fibronectin, fibrin, gelatin, poly gelatin, extracellular matrix (ECM) proteins or peptides, ECM-like proteins or peptides, and combinations thereof.

### Design and Fabrication of the Human Alveolar Interstitium Chip

The functions of lung alveoli are profoundly affected by alveolar anatomical and physiological characteristics. Anatomically, the alveolar wall is a thin epithelium that faces the alveolar lumen and is covered with a thin fluid layer to form an ALI. Supported by a microporous basement membrane, the epithelium forms a barrier due to the presence of tight junctions and adherens junctions [21], which prevents access of nanomaterials to the subepithelium [22]. The interstitium surrounding the epithelium displays an interrelated framework of extracellular matrix (ECM) proteins such as collagen and elastin and is rich with interstitial fluids [23]. In vivo studies suggest that the penetration of nanomaterials into the lung interstitium and interaction with local fibroblasts may be a critical step in nanomaterial-induced fibrogenesis [24]. A key cellular mechanism of fibrogenesis is fibroblast activation and subsequent induction of ECM (in particular, collagen) production and accumulation leading to fibrosis [25, 26]. Lung fibroblasts exhibit the stiffness-dependent fibrogenic responses to MWCNTs [27]. In addition, interstitial fluid flow could alter cytoskeletal organization, influence cell proliferation, and remodel the interstitial matrix [28, 29]. Moreover, the breathing movements expose the epithelium and interstitium to cyclic 3D stretching and relaxation. The mechanical stretch is known to have a profound influence on the formation and function of cells, tissues and organs [30].

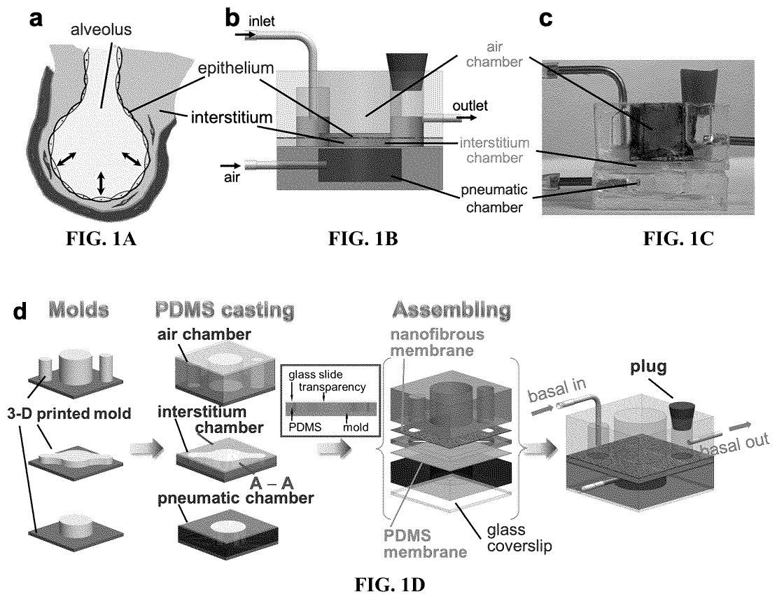

To establish the primary functions of lung alveoli, the inventors developed a dynamic human lung alveolar interstitium-on-a-chip (interstitium chip) system which closely replicates the key anatomical (nanofibrous membrane to imitate the basement membrane) and physiological (interstitial matrix stiffness, interstitial flow, and cyclic 3D breathing-like mechanical stretch) characteristics of human lung alveoli (FIGS. 1A to 1D).

The chip includes a co-culture section and a pneumatic section, both made of, e.g., a water-impermeable material such, e.g., polydimethylsiloxane (PDMS). The co-culture section was comprised of an air (apical) chamber and an interstitium (basal) chamber, between which an electrospun nanofibrous membrane (˜10 μm thick) was sandwiched (FIG. 1D). Human lung alveolar epithelial cells formed a tight epithelium on the nanofibrous membrane at the ALI in the air chamber. Human lung fibroblasts were embedded in a 3D collagenous matrix in the interstitium chamber. The pneumatic section was formed by permanently bonding a PDMS membrane (15 μm thick) on the pneumatic chamber.

The stiffness of the interstitial matrix was primarily a consequence of using a type I collagen (Col I) gel for which the stiffness of the gel can be controlled, with values ranging between 1 kPa and 100 kPa, by adjusting the concentration of Col I solution between 3 and 60 mg/mL, respectively [31]. This range of stiffness covers normal (1-5 kPa) and fibrotic (20-100 kPa) human lung tissues [32-34]. A syringe pump was used to perfuse culture media continuously into the interstitium chamber to generate an interstitial flow of physiological relevance [35-38], as demonstrated by the inventors using a computational simulation.

The inventors had previously determined that physiologically relevant cyclic 3D mechanical stretch significantly upregulated the expression of tight and adhesion junction proteins compared to ID and 2D stretches and static controls [17]. A circular water-impermeable PDMS membrane was thus designed for the pneumatic chamber to provide 3D stretch. When a fluid such as air was pumped in and withdrawn from the pneumatic chamber, the PDMS membrane, the interstitium, and the nanofibrous membrane moved up and down, it generated a 3D radial stretch to mimic breathing movements. However, any method for mechanical stretching can be used such as pneumatic, hydraulic, mechanical, electromechanical, piezo-electric, and the like can be used to stretch the membrane to mimic breathing movements. There was no significant difference in the strain between the experimental observation and the theoretical calculation. Thus, the mechanical stretch was transmitted through the interstitium to the nanofibrous membrane, which exposed the fibroblasts in the interstitium and the epithelial cells on the nanofibrous membrane to the designated strain. The level of applied strain ranges from 5% to 15% at a frequency of 0.2-0.3 Hz (12-20 time/min) to match normal levels of strain observed in alveoli within the whole human lung in vivo [39]. These anatomical (nanofibrous membrane) and physiological (interstitium matrix stiffness, interstitial flow, and cyclic 3D stretch) characteristics of the interstitium chip could be adjusted and optimized to promote and sustain chip function. In addition, the air chamber, electrospun nanofibrous membrane, and interstitium chamber were assembled into the co-culture section by applying the microtransfer assembly (μTA) technique that was previously developed by the present inventors based on microcontact printing with PDMS prepolymer thin film as the adhesive [40]. This technique allowed the inventors to adjust the adhesive strength between the chip sections by controlling the extent of curing of the PDMS thin film which then allowed for the selective separation of sections for downstream analysis.

### Nanofibrous Membrane Promoted the Formation of a Tight Epithelium

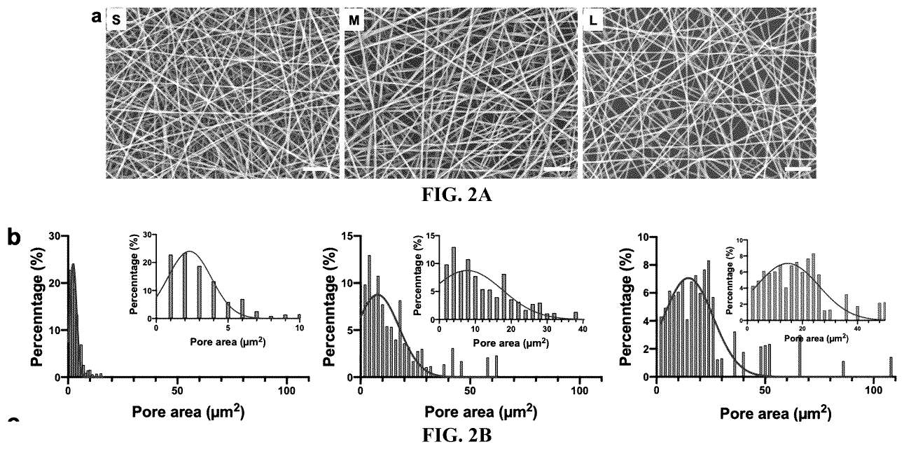

Nanofibrous membranes of various pore sizes were generated by electrospinning polycaprolactone (PCL) solution. By controlling the electrospinning time, nanofibrous membranes of three different pore sizes were fabricated, i.e., small pores (S, 2.32±1.52 μm²), medium pores (M, 8.75±5.79 μm²), and large pores (L, 15.63±9.79 μm²) (FIGS. 2A to 2C). Because all other processing parameters were kept unchanged, the fiber diameters of all of these membranes were similar, i.e., 890±230 nm, 880±240 nm, and 880±210 nm for the small, medium, and large pore size membranes, respectively. Therefore, these membranes allowed us to investigate the effects of pore size on epithelium formation.

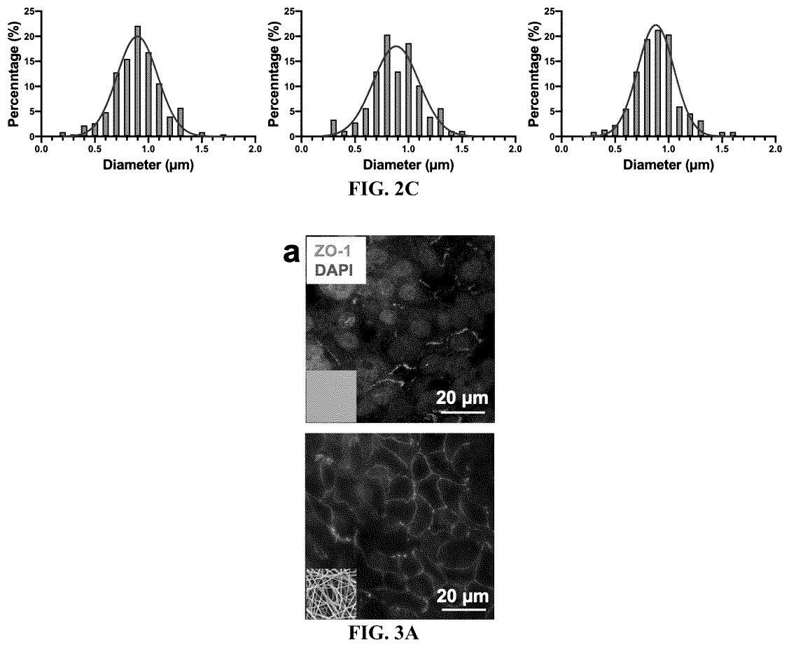

Human alveolar epithelial A549 cells were grown on these nanofibrous membranes while using a PCL flat surface as the control. After 10 days, the epithelial cells displayed continuous tight junction protein zonula occludens (ZO)-1 on the nanofibrous membranes compared to discrete ZO-1 expression on the flat control (FIG. 3A). The pore size effects on the expression of tight junction proteins ZO-1, ZO-3, and occludin and adhesion junction protein E-cadherin were evaluated by western blot. In general, nanofibrous membranes upregulated protein expression and the cells showed significantly higher expressions of ZO-1 and occludin on the medium and large pore membranes compared to the flat control (FIG. 3B). FIG. 3C is a photo of transwell inserts attached with the nanofibrous membrane.

In addition, the nanofibrous membranes were attached to the transwell insert and the permeability coefficient of dextran—commonly used to assess transport via the paracellular route through tight junctions across the epithelial monolayers was assessed. Similarly, the epithelium permeability of the nanofibrous membranes of medium and large sizes were significantly lower than that of the small pore membrane (FIG. 3D).

The basement membrane of lung alveolar epithelium exhibits oval-shaped pores of 0.75 to 3.85 μm in size and the microporous membrane facilitates type II pneumocytes to form a monolayer, enable heterocellular crosstalk, and act as a route for immune cells to move between the epithelium and interstitium [21, 42, 43]. PCL fibrous membranes have been shown to promote the barrier function of lung alveolar epithelium [44]. However, it was unclear if the pore size of fibrous membranes influenced alveolar epithelium barrier function. In this study, PCL nanofibrous membranes with various pore sizes were fabricated and their promoting effects on epithelium formation and permeability were verified. Occludin is linked via zonula occludens protein complexes to tight junctions that seal neighboring epithelial cells and limit paracellular diffusion. The upregulated expression of occludin and ZO-1 proteins and enhanced epithelial permeability on the medium and large pore membranes suggested that both nanofibrous membranes promoted the formation of tight alveolar epithelium. Although the large pore membrane showed a better promoting effect on tight epithelium, there was occasional leakage due to some large pores. Thus, the nanofibrous membrane of medium pores (8.75±5.79 μm²) was selected for the following studies.

### Alveolar Environmental Factors Enhanced the Epithelial Barrier Function

Next, the inventors constructed the interstitium chip with the nanofibrous membrane of medium pore size, constrained by the other key physiological parameters, namely, the interstitium matrix stiffness, interstitial fluid, and cyclic 3D mechanical strain. Collagen is the most abundant protein in the interstitium matrix and thus Col I gel was used as the interstitial matrix for 3D culturing of the normal human lung fibroblasts (NHLFs) on the chip. Our previous study highlighted the significance of matrix stiffness in the fibrogenic responses of lung fibroblasts to MWCNTs [27]. On the chip the concentration of Col I was adjusted to 3 mg/mL to form a gel of 1 kPa in stiffness to resemble the stiffness of normal lung tissue [33]. The interstitial medium perfusion rate was set as 20 μl/hr, which generated an average interstitial fluidic velocity of 0.6-1.4 μm/s, within the physiological range, i.e., 0.1-4.0 μm/s [37, 38]. To replicate the cyclic strain that lung alveoli experience during breath movement, which is 5-15% linear strain at a frequency of 0.2-0.3 Hz in normal conditions, a theoretical maximum 15% linear strain was applied at a frequency of 0.2 Hz. For comparison, the conventional transwell model was built as the controls. In the transwell model, the epithelial and fibroblast cells were grown on the apical and basal side of the insert membrane, respectively.

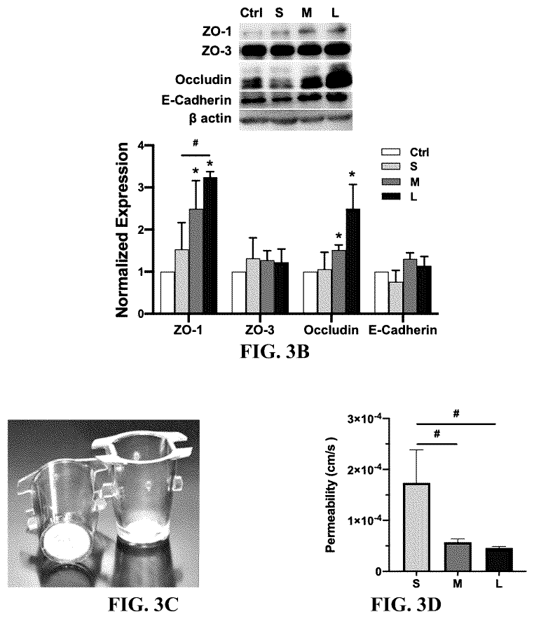

After one week of culture, the epithelial cells formed a dense epithelial layer on the chip; however, the gaps between the epithelial cells were evident in the transwell model (FIG. 4A, 4B). The epithelium permeability was evaluated using dextrans of 4 kDa and 70 kDa in molecular weight, which represents the intermediate and large-size agents transported through tight junctions [41, 46]. As shown in FIG. 4C, the permeability of both dextrans through cells grown on the chip was about 3˜4 fold lower than that of the transwell model, i.e., 2.16±0.51×10⁻⁵ vs. 6.12±2.58×10⁻⁵ cm/s for 4 kDa dextran, and 7.79±1.75×10⁶ vs. 2.97±1.21×10⁻⁵ cm/s for 70 kDa dextran. Note that the epithelium permeability of 4 kDa dextran on the chip was also improved by 2-fold compared to the monoculture on the nanofibrous membrane, i.e., 2.16±0.51×10⁻⁵ vs. 5.73±2.70×10⁻⁵ (FIG. 3D, Table 1).

TABLE 1
Permeability Summary.

| | | Permeability (cm/s) | |
|---|---|---|---|
| Model | Duration | 4 kDa dextran | 70 kDa dextran |
| Interstitium chip | 4 weeks | 7.08 ± 1.24 × 10⁻⁶ | 9.77 ± 0.84 × 10⁻⁸ |
| | 1 week | 2.16 ± 0.51 × 10⁻⁵ | 7.79 ± 1.75 × 10⁻⁶ |
| Transwell co-culture | 4 weeks | 4.96 ± 3.39 × 10⁻⁵ | 1.73 ± 0.40 × 10⁻⁶ |
| | 1 week | 6.12 ± 2.58 × 10⁻⁵ | 2.97 ± 1.21 × 10⁻⁵ |
| Nanofibrous membrane only | 1 week | 5.73 ± 2.73 × 10⁻⁵ | — |
| Literature reports [41, 47, 48] | | 10⁻⁶-10⁻⁵ | 10⁻⁷-10⁻⁶ |
| Interstitium chip | 4 weeks | 7.08 ± 1.24 × 10⁻⁶ | 9.77 ± 0.84 × 10⁻⁸ |
| | 1 week | 2.16 ± 0.51 × 10⁻⁵ | 7.79 ± 1.75 × 10⁻⁶ |
| Transwell co-culture | 4 weeks | 4.96 ± 3.39 × 10⁻⁵ | 1.73 ± 0.40 × 10⁻⁶ |
| | 1 week | 6.12 ± 2.58 × 10⁻⁵ | 2.97 ± 1.21 × 10⁻⁵ |
| Nanofibrous membrane only | 1 week | 5.73 ± 2.73 × 10⁻⁵ | — |
| Literature reports [41, 47, 48] | | 10⁻⁶-10⁻⁵ | 10⁻⁷-10⁻⁶ |

In brief, the biomimetic interstitium chip demonstrated enhanced epithelial barrier function compared to a conventional transwell model and monoculture on the nanofibrous membrane. This enhancement was likely attributed to the synergy of the nanofibrous membrane, co-culture of the epithelial and fibroblast cells in a 3D gel, and physiologically relevant interstitium matrix stiffness, interstitial flow, and cyclic 3-D stretch.

### The Col I-Fibrin Interstitial Matrix Sustained the Chip

After two weeks of culture, interstitial matrix degradation and remodeling was observed on the chip. It is known that collagenous gels degrade relatively fast, and fibroblasts caused collagen gel contraction [49]. To alleviate the interstitium deterioration, fibrin was utilized to reinforce the collagen gel because fibrin gels have been reported to be stable for at least 12 months [50]. The degradation of fibrin gels, a process called fibrinolysis, can be controlled by inhibitors such as ε-aminocaproic acid (EACA) [51]. Additionally, fibrin gels can facilitate cell adhesion and promote fibroblasts to synthesize more collagen and other ECM proteins compared to collagen gels [52, 53]. Compared with pure collagen and fibrin gels, the Col I-fibrin blend gels exhibit intermediate properties [54]. Therefore, Col I-fibrin blend gels were prepared by mixing Col I solution and fibrin solution at volume ratios from 1:0.1 to 1:1 for 3D culturing of the lung fibroblasts. After culture under static conditions for two weeks, no gel deformation was observed in all blend gels, the fibroblasts spread, and cell elongation increased with an increase in the fibrin concentration. The observation indicated that adding fibrin improved the interstitium integrity and likely extended chip longevity. To maintain matrix stability while maximizing collagen content, a Col I-fibrin gel with a ratio of 1:0.3 was used for long-term culture of the chip.

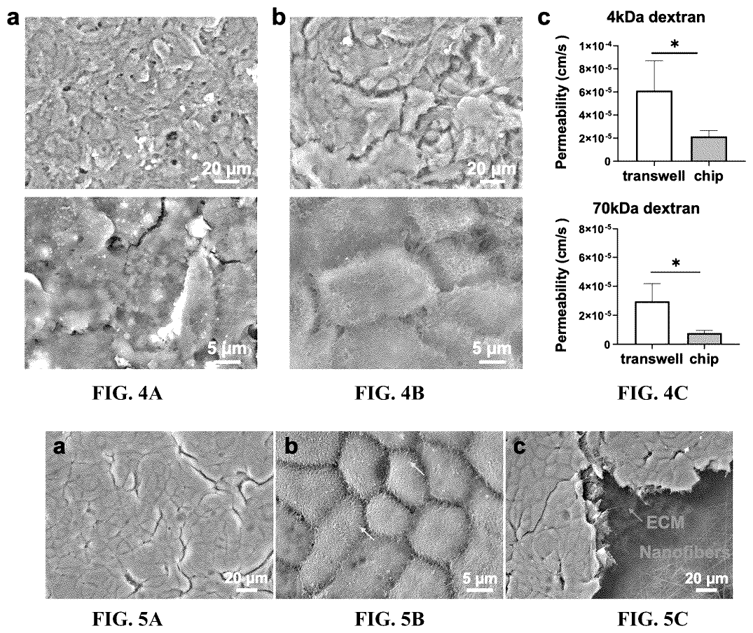

The epithelial cells formed a monolayer after one week of culture on the chip with Col I fibrin gel as the interstitial matrix. The epithelial cell growth medium was then aspirated from the air chamber to form an ALI and a mixed epithelial and fibroblast culture media (1:1, v/v) was perfused into the interstitium chamber. Three weeks later, a tight epithelium formed and the A549 cells displayed cuboidal shape and presented apical microvilli—the typical ultrastructural feature of human alveolar epithelial type II (ATII) cells (FIGS. 5A, 5B). The layered structure of epithelium, secreted ECM, and electrospun nanofibrous membrane was evident in FIG. 5C. Importantly, the epithelium permeability was further enhanced. For example, for 4 kDa dextran, the permeability decreased by 3-fold from 2.16±0.51×10⁻⁵ cm/s (1 week) to 7.08±1.24×10⁻⁶ cm/s (4 weeks), significantly lower than 4.96±3.39×10⁻⁵ cm/s for the transwell model. For 70 kDa dextran, the permeability decreased by about 80-fold, from 7.79±1.75×10⁻⁶ cm/s (1 week) to 9.77±0.84×10⁻⁸ cm/s (4 weeks) compared to 1.73±0.40×10⁻⁶ cm/s for the transwell model (Table 1, FIG. 5D). The enhanced epithelium permeability values were comparable or lower than literature reports using transwell models and microfluidic devices [41, 47, 48].

A549 cells feature some of the ATII cell properties and are often used in in vitro lung alveolar models [47]. It was reported that ATII cells cocultured in transwells with lung fibroblasts embedded in a 3D matrix maintained their distinct phenotype for 7 days; conversely, the cells lost their phenotype within 3-5 days on conventional 2D models [55, 56]. On this interstitium chip, the A549 cells formed a tight epithelium in one week and displayed distinct ATII cell structural features like cuboidal shape and apical microvilli. Moreover, the epithelium permeability was significantly lower than the transwell models. Of note, no evident deterioration of the chip was observed after 8-weeks of culture; conversely, individual epithelial cells instead of a continuous cell monolayer was observed in the transwell model.

The biomimetic interstitium chip demonstrated an enhanced epithelial barrier function with extended longevity. As fibrin is known to be formed by enzymatic cleavage and reorganization of fibrinogen and thrombin [57], interstitium integrity can be enhanced by reducing gel degradation via the administration of EACA, which blocks the binding of plasmin or plasminogen to fibrin [51].

A549 cells provide a reliable and straightforward cell source for chip development. However, the cells are from a lung tumor, are likely genetically unstable, and might not fully recapitulate epithelial barrier function in vivo. The use of primary human alveolar epithelial cells or epithelial basal stem cells will facilitate the establishment of a more in vivo like model [58]. Human macrophages can also be co-cultured with the epithelial cells at the ALI to facilitate the maintenance of lung homeostasis [59]. Taken together, the key alveolar microenvironmental factors can be fine-tuned to further improve chip longevity and performance.

### CNT Toxicity Within the Biomimetic Interstitium Chip was Consistent With In Vivo Observations

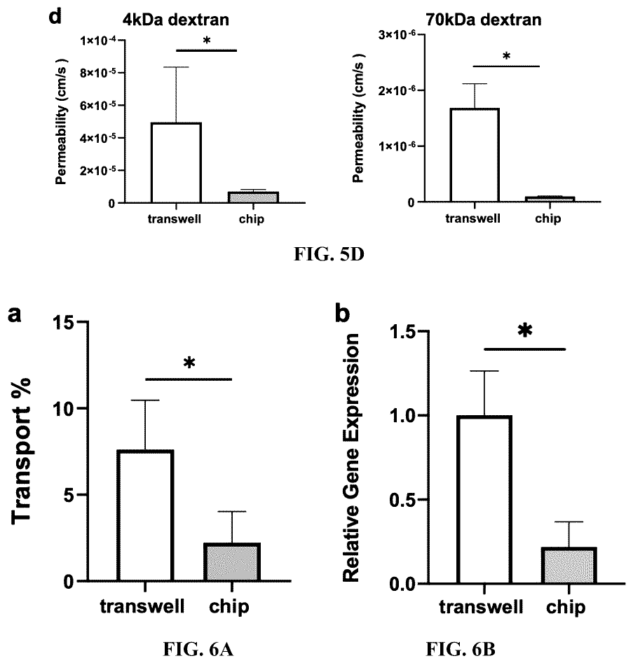

Inhaled nanoparticles have been shown to pass through the epithelium barrier to the subepithelium and induce lung diseases by stimulating profibrotic responses and proinflammatory cytokine and chemokine secretion [60, 61]. Therefore, the potential application of this lung interstitium chip for nanotoxicity studies were explored. Of interest were MWCNTs because MWCNTs have been extensively assessed both in vivo and in vitro [20, 62, 63], which provided a benchmark for chip validation. The transport of MWCNTs through the epithelium and the lung inflammatory response were monitored after the epithelium was exposed to MWCNTs (5 μg/ml) for 24 hours. The percentage of MWCNTs that crossed the epithelium into the interstitium on the chip was 2.24±1.80%, significantly lower than the 7.61±2.85% seen in the transwell model (FIG. 6A). This value is consistent with in vivo observations [20]. The culture medium was collected from the interstitium chamber and multiple inflammatory chemokines were measured. Although the expression of interleukin-4 (IL-4) and IL-6 was too weak to be detected, IL-8 expression associated with the fibroblasts was lower on the chip than the transwell model (FIG. 6B). The interstitium chip exhibited enhanced epithelial barrier function compared to the transwell models. As such, the penetration of MWCNTs across the epithelium was substantially reduced, and subsequently, the inflammatory responses of the fibroblasts to MWCNTs were alleviated, being in line with previous reports [20, 64, 65].

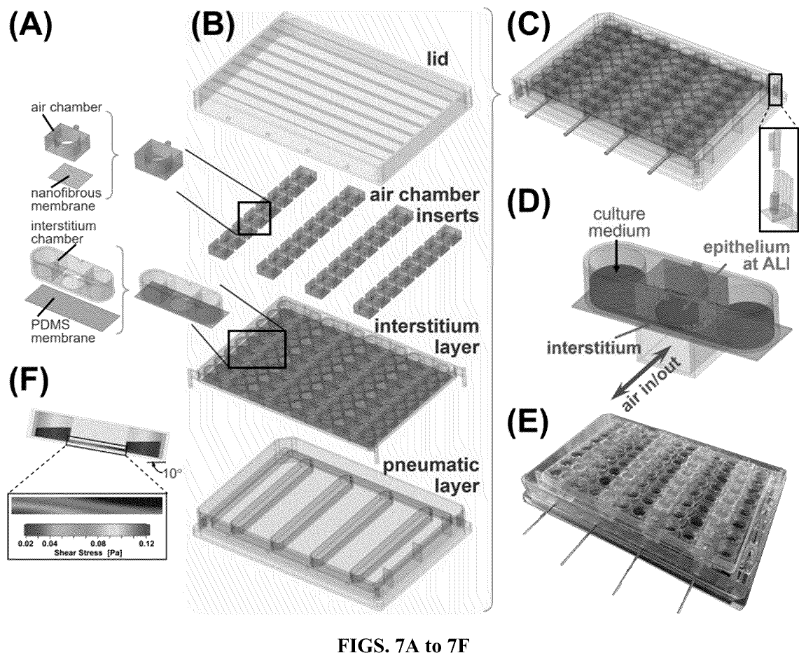

The single chip will be expanded to a 32-chip array in a 96-well format (FIGS. 7A to 7F). The array includes a lid, air chamber inserts (4 pieces, each has 8 inserts), an interstitium layer (4×8 interstitium chambers), and a pneumatic layer (FIG. 7B). These parts are printed using a 3-D printer (Form 3+; Formlabs Co). The bottoms of the air chamber inserts and interstitium layer are attached with electrospun nanofibrous membranes (˜20 μm thick) and PDMS (Sylgard 184) membranes (˜50 μm), respectively (FIG. 7A), by using PMDS thin film adhesive. The fibroblast-laden Col I/fibrin gels are loaded in the interstitium chamber, and the air chamber inserts are placed in the middle of the interstitium chamber, followed by the epithelial cell seeding on the nanofibrous membrane in the air chamber. This chip array adopts a 96-well format and thus enables us to use automated liquid handling tools to load the cell-laden interstitium gel, seed epithelial cells, and add culture media. Upon ALI formation, the epithelial culture medium will be aspirated from the air chamber and only mixed epithelial/fibroblast media will be added into the interstitium. Human macrophages will be cultured with epithelial cells, macrophages and neutrophils will be mixed and added into interstitium to imitate the immune reaction.

The assembled multi-chip array will be placed on a rocker to generate interstitial flow in the physiological range (FIGS. 7B, 7C, 7D, and 7E). The interstitium flow can be analyzed by SolidWork simulation (exemplified in FIG. 7F) and verified by measuring the movement of fluorescent NPs. Moreover, a syringe pump is used to pump air in/out of the pneumatic layer to provide cyclic, 3D stretch of 5-15% in the strain at a frequency of 12-20 time/min. The applied strain can be verified by imaging using a charge-coupled device (CCD) camera and analyzed using ImageJ. The cell health, epithelium integrity, and barrier function, interstitium integrity, ROS production, and omics analysis. A comparison between the single chip and the multi-chip array ensures that the chips on the array function in the same way as the single chip.

It was found that the lung alveolar interstitium chip described herein closely imitated the key anatomical (epithelial cells co-cultured with fibroblasts encapsulated in 3D collagenous interstitial matrix via nanofibrous membrane) and physiological (interstitium matrix stiffness, interstitial flow, and 3D breathing-like mechanical stretch) characteristics of a human lung alveolus. The electrospun nanofibrous membrane promoted the formation of tight epithelium. Overall, the biomimetic interstitium chip demonstrated enhanced epithelial barrier function and extended longevity beyond 8 weeks with Col I-fibrin blend gels as the interstitium matrix. The key alveolar microenvironmental factors, such as nanofibrous membrane and interstitial matrix, can be optimized to maintain the homeostasis of the epithelium and interstitium and thus further extend chip longevity. Importantly, the toxicity assessment of MWCNTs on the chip verified that the biomimetic interstitium chip represented a useful in vitro model for human lung alveolar interstitium. The interstitium chip system can be used for drug development, disease modeling, and high throughput screening of drugs.

### Materials and Methods. Cell Culture

Human alveolar epithelial cells (A549; Cat #: CCL-185, ATCC, Manassas, VA, USA) were cultured in Dulbecco's Modified Eagle Medium (DMEM) with L-glutamine (Life Technologies, Carlsbad, CA, USA) supplemented with 10% fetal bovine serum (FBS; Sigma-Aldrich, St Louis, MO, USA), 100 U/ml penicillin and 100 μg/ml streptomycin (Life Technologies). Normal human lung fibroblasts (NHLFs; Cat #: CC-2512, Lonza, Walkersville, MD, USA) were cultured in fibroblast basal medium (Lonza) supplemented with FGM-2 SingleQuots supplements (Lonza), 100 U/ml penicillin, and 100 μg/ml streptomycin.

### Fabrication and Characterization of Nanofibrous Membranes

Nanofibrous membranes were fabricated by electrospinning a solution of PCL in 1,1,1,3,3,3-hexafluoro-2-propanol (HFIP, 10%, w/v). Briefly, the PCL solution was loaded in a syringe with a blunt-tipped needle as the spinneret and connected to a syringe pump. A 4″×4″ square glass plate was placed on a 3″×3″ aluminum-covered plate as a collector, which was placed 16 cm below the needle tip. The needle tip was connected to a 14 kV voltage and the aluminum-covered plate was grounded. The PCL solution was ejected at a flow rate of 0.5 ml/hour and the nanofibers were deposited on the collector.

The formed nanofibrous membranes were sputter-coated with gold using Denton Vacuum Desk V sputter coater (Denton Vacuum, Moorestown, NJ, USA) and imaged using a scanning electron microscope (SEM; TM3030 Plus, Hitachi High-Technologies Co., Tokyo, Japan). The fiber diameter and membrane pore size were analyzed using ImageJ (http://rsb.info.nih.gov/ij/index.html). For the fiber diameter, a line was drawn across the fiber, and then the length was measured. For the pore size, the images were adjusted using the threshold command and the area of each pore was analyzed using the analyze particles function. A histogram line graph was then generated to show the distribution of fiber diameters or pore sizes.

### Fabrication of the Lung Interstitium Chip

The chip consisted of an upper air chamber, a middle interstitium chamber, and a lower pneumatic chamber (FIGS. 1A to 1D), which had a concentric circle of 10 mm in diameter and different heights of 10, 2, and 5 mm, respectively. All the chambers were prepared by casting a mixture of PDMS resin and curing agent at a w/w ratio of 10:1.05 (Sylgard 184, Ellsworth Adhesives, Germantown, WA, USA) on their corresponding 3D printed mold and curing them at 75° C. for 2 hours. To obtain the interstitium chamber with an open structure, the PDMS mixture was sandwiched between the 3D mold and a transparency film with a glass slide under a compressive pressure of approximately 1 MPa (illustrated in the red box in FIG. 1D). The pneumatic chamber was permanently bonded with a thin PDMS membrane via a μTA technique that were previously developed by the present inventors [40]. Briefly, the thin PDMS membrane was prepared by spin-coating a PDMS mixture on a silicon wafer at 2500 rpm for 1 min and curing it at 75° C. for 1 hour. The pneumatic chamber with the opening facing down was then stamped on a thin PDMS layer, which was spin-coated on a silicon wafer at 4000 rpm for 30 s, and transferred onto the thin PDMS membrane, followed by curing at 75° C. for 1 hour under a compressive pressure of approximately 1 MPa. Subsequently, the pneumatic chamber with the PDMS membrane was gently peeled off from the silicon wafer and was bonded with the middle interstitium chamber reversibly by applying the μTA technique again.

For the reversible bonding, first, a 33% solution of PDMS mixture in hexane (Thermo Fisher Scientific, Waltham, MA, USA) was spin-coated on a silicon wafer at 4000 rpm for 2 minutes to form a thin adhesive layer, followed by prebaking at 50° C. for 5 minutes. Secondly, the interstitium chamber was stamped on the adhesive layer for 30 seconds and then transferred onto the pneumatic chamber, followed by curing at 75° C. for 1 hour under a compressive pressure of approximately 1 MPa. Next, the nanofibrous membrane was reversibly bonded between the interstitium and air chambers following the aforementioned bonding process. One hole was punched in the pneumatic chamber and connected via the stainless-steel tubing to a programmable syringe pump (PHD 2000, Harvard Apparatus, Holliston, MA, USA), which provided cyclic mechanical stretch. Two holes were made in the air chamber as basal in and out ports for the culture medium perfusion through the interstitium chamber.

### Characterization of Mechanical Strain

The mechanical strain (deformation) of the PDMS membrane on the pneumatic chamber with and without the interstitium layer was characterized using a previously described method [17]. Briefly, the deformation of the membrane or the membrane with interstitium layer was imaged using a charge-coupled device (CCD) camera (Model DMK 31; The Imaging Source, Charlotte, NC, USA). The captured images were adjusted using ImageJ to get a curvilinear profile, which was then digitalized using GetData Graph Digitalizer (www.getdata-graph-digitalizer.com). The measured linear strain was compared with the theoretical calculation as previously described [17].

### Transwell Culture Models

Two transwell models were prepared for epithelial cell monoculture and co-culture as follows. Transwell monoculture models were prepared by attaching the nanofibrous membrane onto a transwell insert using the μTA technique described in Fabrication of the lung interstitium chip (above), and the epithelial cells were cultured on the apical side of the membrane. Transwell co-culture models were prepared as the control for the interstitium chip studies. NHLF at a seeding density of 2×10⁵ cells/cm² were seeded onto the basal side of the transwell insert membrane (0.6 μm; Sterlitech, Auburn, WA, USA) and incubated for 1 hour. The insert was then inverted and A549 cells at 2×10⁵ cells/cm2 were placed in the upper compartment. For the ALI culture, the epithelial cell culture medium was removed after one week from the insert and the mixed epithelial and fibroblast culture media (1:1; v/v) was added to the low compartment and changed every three days.

### Cell Culture on Lung Interstitium Chips

The chip was first oxygen plasma treated at the medium power for 1 min in a plasma cleaner (Model PDC-001; Harrick Plasma, Ithaca, NY, USA) and sterilized with 70% ethanol for 1 h, followed by rinsing with phosphate buffered saline (PBS, Sigma-Aldrich). Then, 500 μl of 50 μg/ml Col I solution (rat tail; Corning, Corning, NY, USA) was added to the cell culture chamber and interstitium chamber and incubated overnight in an incubator for the surface coating. The interstitium layer was prepared with Col I or Col I-fibrin blend hydrogel. In the case of Col I gels, Col I solution was prepared by mixing Col I, 10×PBS, 1 N sodium hydroxide, and DMEM following the manufacturer's protocol to obtain a final Col I concentration of 3 mg/ml. In the case of Col I-fibrin blend gels, fibrin solution was prepared by mixing solution A (a mixture of bovine fibrinogen (Sigma-Aldrich) and ε-aminocaproic acid (EACA, Sigma-Aldrich)) and solution B (a mixture of bovine thrombin (Sigma-Aldrich) and calcium chloride (CaCl₂, Sigma-Aldrich)), with a final concentration of 5 mg/ml fibrinogen, 2 U/ml thrombin, and 5 mM CaCl₂, and 2 mg/ml EACA. Subsequently, the fibrin solution was mixed with Col I solution (3 mg/ml) and human lung fibroblasts (at a seeding density of 2.0×10⁵ cells/mL), and the mixture was injected into the interstitium chamber and incubated at 37° C. for 4 hours to form the cell-laden hydrogel matrix. The A549 cells at a density of 2.0×10⁵ cells/cm₂ were then added into the air chamber and cultured under static conditions for three days before the interstitial flow and cyclic mechanical stretch were applied. After being cultured for one week under the mechanical stretch, the epithelial cell culture medium was aspirated from the air chamber and a mixed epithelial and fibroblast culture media (1:1, v/v) supplemented with EACA (2 mg/ml) was perfused through the interstitium chamber.

### Immunofluorescent Staining

The cells were fixed with 1:1 (v/v) of methanol: acetone at −20° C. for 10 min and blocked with tris buffered saline (TBS) (KD Medical, Columbia, MD, USA) containing 10% (v/v) FBS, 10% (v/v) goat serum (Sigma-Aldrich), and 1% bovine serum albumin (BSA; Akron Biotech, Boca Raton, FL, USA) at room temperature for 30 minutes. After blocking, the cells were incubated with primary antibody at 4° C. overnight followed by secondary antibody incubation at room temperature for 1 hour. The nuclei were stained with SlowFade™ Gold Antifade Mountant with DAPI (Life Technologies) and the samples were examined using a Nikon Ti eclipse fluorescence microscope (Nikon, Melville, NY, USA).

### Western Blotting

The total proteins were extracted by lysing the cells with radioimmune precipitation assay (RIPA) buffer (Santa Cruz Biotechnology, Santa Cruz, CA, USA) that contained protease inhibitor for 30 min on ice followed by centrifuging at 4° C., 12,000 rpm for 5 minutes and collecting the supernatant. Proteins were separated by 8% sodium dodecyl sulfate-polyacrylamide gel electrophoresis (SDS-PAGE) and transferred onto polyvinylidene fluoride (PVDF) membrane (Thermo Fisher Scientific). The membranes were blocked in 5% nonfat milk (RPI International, Mount Prospect, IL, USA) dissolved in TBS with 0.1% Tween-20 (Thermo Fisher Scientific) at room temperature for 1 hour. Subsequently, the membrane was incubated with primary antibody and horseradish peroxidase (HRP)—conjugated secondary antibody. Protein bands were visualized by ECL detection kit (EMD Millipore, Burlington, MA, USA) and images were acquired using the ChemiDoc MP image system (Bio-Rad, Hercules, CA, USA). The band intensity was quantified using ImageJ and normalized to the expression of β-actin.

### Permeability Assay

The epithelial permeability was measured in the 12-well transwell plate-based models including the epithelial cell monoculture on a nanofibrous membrane and co-culture with fibroblast cells and on the interstitium chips. In the transwell models, 500 μl of 4 kDa (100 μg/ml) or 70 kDa (500 μg/ml) fluorescein isothiocyanate (FITC)-dextran (Sigma-Aldrich) was added to the insert and 1 ml of cell culture medium was added to the low compartment. The fluorescence intensity of the medium from the insert and the low compartment was measured after incubation for 2 hours. On the chip, the interstitium perfusion was halted and 200 μl of FITC-dextran was added into the air chamber. The chip was incubated for 2 hours in an incubator and then the gel and medium in the interstitium were collected after the chip was dissembled for fluorescence intensity measurements.

The concentration of FITC-dextran was determined by fluorescence intensity that was compared to a calibration curve of FITC-dextran concentration vs fluorescence intensity. The apparent permeability Pₐₚₚ was calculated as

$${P}_{app} = \frac{{C}_{b} {V}_{b}}{tA Δ C} ,$$

where C_{b} and V_{b} are the dextran concentration and solution volume in the low compartment of the transwell or interstitium chamber of the chip, respectively, t is the diffusion time, A is the total area of diffusion, ΔC is the concentration change across the barrier [66]. The permeability coefficient of the epithelial barrier, Pₑₚᵢ, was determined from the Pₐₚₚ and the background permeability coefficient Pₒ (measured in a transwell plate or chip without epithelium) as follows:

$$\frac{1}{{P}_{ept}} = \frac{1}{{P}_{app}} - \frac{1}{{P}_{o}} ( s / cm ) .$$

### SEM Imaging

The epithelial cells were fixed in situ with 4% paraformaldehyde (PFA, Sigma-Aldrich) and 2% glutaraldehyde solution (Fisher Chemical, Fairlawn, NJ, USA) at room temperature for 4 hours and then dehydrated with gradient ethanol (30%, 50%, 70%, 80%, 90%, 95%, and 100%) and then hexamethyldisilazane (HMDS), each step being 10 minutes. The epithelial cell layer on the nanofibrous membrane or the transwell membrane was taken off, sputter-coated by gold, and imaged as detailed in the Fabrication and Characterization of nanofibrous membranes (above).

### Carbon Nanotubes (CNT) Penetration Assay

Multi-Walled Carbon Nanotubes (MWCNTs) (XNRI MWNT-7; Mitsui & Company, NY, USA) were dispersed in PBS containing 5 mg/ml of BSA and diluted in culture medium to a concentration of 5 μg/ml as previously reported [27]. The MWCNT suspension was added to the transwell insert or the chip air chamber, similar to the permeability assay in 4.9 with the MWCNT suspension replacing the FITC-dextran. After incubation at 37° C. for 24 hours, the medium in the transwell low compartment was collected for optical density (OD) measurement. The gel and medium in the interstitium chamber of the chip were collected and digested with 4 mg/ml collagenase in PBS at 37° C. before the OD measurement. The OD 640 value of the solution was measured and MWCNT concentration was calculated by normalizing the OD value with the value obtained from the standard curve and thus the relative transport across the barrier was calculated.

### Real-Time Quantitative Reverse Transcription-Polymerase Chain Reaction (qRT-PCR) Assay

After MWCNT treatment, the expression of transcripts for the pro-inflammatory cytokines. IL-4, 6, 8 derived from the NHLFs on the chip and transwell cultures were analyzed by qRT-PCR assay. The NHLF in the interstitium of the chip was collected by incubating with 4 mg/ml collagenase at 37° C. and centrifuged for 5 minutes at 400 g at 4° C. Total RNA was extracted using the Aurum Total RNA Mini Kit (Bio-Rad) and cDNA was synthesized using iScript RT Supermix (Bio-Rad). The qRT-PCR samples were prepared using SsoAdvanced Univ SYBR Grn Suprmix (Bio-Rad). The reaction was performed using a CFX96 Touch Real-Time PCR Detection System (Bio-Rad) under the following conditions: 40 cycles of 95° C. for 15 s, 60° C. for 30 s, followed by a melt curve of 65-95° C. at 0.5° C. increments, at 2-5 sec/step. mRNA expression was normalized to α-tubulin mRNA expression.

### Statistical Analysis

The data were presented as mean±standard error of the mean (S.E.M.). The statistical differences were analyzed using two-tailed t test using Prism 8 (GraphPad software, San Diego, CA, USA). Statistically significant differences were considered at a level of p<0.05.

As embodied and broadly described herein, an aspect of the present disclosure relates to a 3D microfluidic device mimicking an anatomy and physiology of a human lung alveolar interstitium comprising, consisting essentially of, or consisting of: a housing defining a cavity, at least one inlet and one outlet, wherein the inlet and outlet are in fluid communication with an inlet chamber and an outlet chamber; an interstitium chamber positioned in the cavity that comprises an electrospun nanofibrous membrane that supports the growth of lung epithelial cells and fibroblasts, wherein the electrospun nanofibrous membrane is in fluid communication with the at least one inlet chamber and an outlet chamber to provide a growth media to the lung epithelial cells and fibroblasts; a pneumatic chamber separated from the interstitium chamber by a water-impermeable membrane and a source of air in fluid communication with the pneumatic chamber; and an air chamber in fluid communication a second side of the with electrospun nanofibrous membrane of the interstitium chamber, wherein the air chamber is opposite the pneumatic chamber; wherein the integration of inputs and outputs mimics the anatomy and physiology of the human lung alveolar interstitium. In one aspect, the inlet aperture and the outlet aperture allow for injection of at least one of fluid, gas and solid material within and through the device. In another aspect, the device is configured to be filled with cellular components on the electrospun nanofibrous membrane. In another aspect, the microfluidic device further comprises one or more seals that separate the interstitium chamber, pneumatic chamber, and air chamber. In another aspect, the microfluidic device further comprises a pump to provide pneumatic pressure in the pneumatic chamber. In another aspect, the device comprises a material selected from a group consisting of glass, silicon, polysiloxane, polydimethylsiloxane, and optically transparent polymers. In another aspect, the electrospun nanofibrous membrane has at least one of: physiological interstitial matrix stiffness or physiological 3D breathing mechanical stretch. In another aspect, the interstitium chamber further comprises a collagen I-fibrin blend gel. In another aspect, the interstitium chamber comprises one or more lung cell lines, lung primary cell cultures, alveolar epithelial cells, fibroblasts, immune cells or combinations thereof.

As embodied and broadly described herein, an aspect of the present disclosure relates to a lab-on-a-chip comprising a 3D microfluidic device mimicking an anatomy and physiology of a human lung alveolar interstitium comprising, consisting essentially of, or consisting of: a housing defining a cavity, at least one inlet and one outlet, wherein the inlet and outlet are in fluid communication with an inlet chamber and an outlet chamber; an interstitium chamber positioned in the cavity that comprises an electrospun nanofibrous membrane that supports the growth of lung epithelial cells and fibroblasts, wherein the electrospun nanofibrous membrane is in fluid communication with the at least one inlet chamber and an outlet chamber to provide a growth media to the lung epithelial cells and fibroblasts; a pneumatic chamber separated from the interstitium chamber by a water-impermeable membrane and a source of air in fluid communication with the pneumatic chamber; and an air chamber in fluid communication a second side of the with electrospun nanofibrous membrane of the interstitium chamber, wherein the air chamber is opposite the pneumatic chamber; wherein the integration of inputs and outputs mimics the anatomy and physiology of the human lung alveolar interstitium. In one aspect, the inlet aperture and the outlet aperture allow for injection of at least one of fluid, gas and solid material within and through the device. In another aspect, the device is configured to be filled with cellular components on the electrospun nanofibrous membrane. In another aspect, the lab-on-a-chip further comprises one or more seals that separate the interstitium chamber, pneumatic chamber, and air chamber. In another aspect, the lab-on-a-chip further comprises a pump to provide pneumatic pressure in the pneumatic chamber. In another aspect, the device comprises a material selected from a group consisting of glass, silicon, polysiloxane, polydimethylsiloxane, and optically transparent polymers. In another aspect, the electrospun nanofibrous membrane has at least one of: physiological interstitial matrix stiffness or physiological 3D breathing mechanical stretch. In another aspect, the interstitium chamber further comprises a collagen I-fibrin blend gel. In another aspect, the interstitium chamber comprises one or more lung cell lines, lung primary cell cultures, alveolar epithelial cells, fibroblasts, or combinations thereof.

As embodied and broadly described herein, an aspect of the present disclosure relates to a kit comprising a lab-on-a-chip comprising, consisting essentially of, or consisting of: a 3D microfluidic device mimicking an anatomy and physiology of a human lung alveolar interstitium comprising: a housing defining a cavity, at least one inlet and one outlet, wherein the inlet and outlet are in fluid communication with an inlet chamber and an outlet chamber; an interstitium chamber positioned in the cavity that comprises an electrospun nanofibrous membrane that supports the growth of lung epithelial cells and fibroblasts, wherein the electrospun nanofibrous membrane is in fluid communication with the at least one inlet chamber and an outlet chamber to provide a growth media to the lung epithelial cells and fibroblasts; a pneumatic chamber separated from the interstitium chamber by a water-impermeable membrane and a source of air in fluid communication with the pneumatic chamber; and an air chamber in fluid communication a second side of the with electrospun nanofibrous membrane of the interstitium chamber, wherein the air chamber is opposite the pneumatic chamber; wherein the integration of inputs and outputs mimics the anatomy and physiology of the human lung alveolar interstitium.

As embodied and broadly described herein, an aspect of the present disclosure relates to a method mimicking an anatomy and physiology of a human lung alveolar interstitium with a lab-on-a-chip comprising a 3D microfluidic device comprising, consisting essentially of, or consisting of: providing a housing defining a cavity, at least one inlet and one outlet, wherein the inlet and outlet are in fluid communication with an inlet chamber and an outlet chamber; inserting lung epithelial cells and fibroblasts into an interstitium chamber positioned in the cavity that comprises an electrospun nanofibrous membrane that supports the growth of the lung epithelial cells and fibroblasts, wherein the electrospun nanofibrous membrane is in fluid communication with the at least one inlet chamber and an outlet chamber to provide a growth media to the lung epithelial cells and fibroblasts; applying pneumatic pressure to the device into a pneumatic chamber separated from the interstitium chamber by a water-impermeable membrane and a source of air in fluid communication with the pneumatic chamber; and providing an air chamber in fluid communication a second side of the with electrospun nanofibrous membrane of the interstitium chamber, wherein the air chamber is opposite the pneumatic chamber; wherein the integration of inputs and outputs mimics the anatomy and physiology of the human lung alveolar interstitium. In one aspect, the inlet aperture and the outlet aperture allow for injection of at least one of fluid, gas and solid material within and through the device. In another aspect, the device is configured to be filled with cellular components in the electrospun nanofibrous membrane. In another aspect, the method further comprises one or more seals that separate the interstitium chamber, pneumatic chamber, and air chamber. In another aspect, the method further comprises a pump to provide pneumatic pressure in the pneumatic chamber. In another aspect, the device comprises a material selected from a group consisting of glass, silicon, polysiloxane, polydimethylsiloxane, and optically transparent polymers. In another aspect, the electrospun nanofibrous membrane has at least one of: physiological interstitial matrix stiffness or physiological 3D breathing mechanical stretch. In another aspect, the interstitium chamber further comprises a collagen I-fibrin blend gel. In another aspect, the interstitium chamber comprises one or more lung cell lines, lung primary cell cultures, alveolar epithelial cells, fibroblasts, or combinations thereof.

As embodied and broadly described herein, an aspect of the present disclosure relates to a high-throughput method of determining the effectiveness of a candidate drug that impacts lung alveoli, the method comprising, consisting essentially of, or consisting of: providing a housing defining an array of test wells that each comprise a first and a second cavity, each of the cavities comprising at least one inlet and one outlet, wherein the inlet and outlet are in fluid communication with an inlet chamber and an outlet chamber; inserting lung epithelial cells and fibroblasts into an interstitium chamber positioned in the housing that comprises an electrospun nanofibrous membrane that supports the growth of the lung epithelial cells and fibroblasts, wherein the electrospun nanofibrous membrane is in fluid communication with the at least one inlet chamber and an outlet chamber to provide a growth media to the lung epithelial cells and fibroblasts; applying pneumatic pressure into a pneumatic chamber separated from the interstitium chamber by a water-impermeable membrane and a source of air in fluid communication with the pneumatic chamber; and providing an air chamber in fluid communication a second side of the electrospun nanofibrous membrane of the interstitium chamber, wherein the air chamber is opposite the pneumatic chamber; wherein the integration of inputs and outputs mimics the anatomy and physiology of the human lung alveolar interstitium; administering a candidate drug to at least a first test well, and a placebo to at least a placebo test well; and determining if the candidate drug modifies one or more parameters associated with lung function in the at least first test well when compared to the placebo test well over a course of treatment with the candidate drug.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In embodiments of any of the compositions and methods provided herein, “comprising” may be replaced with “consisting essentially of” or “consisting of”. As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step or a limitation) or group of integers (e.g., feature(s), clement(s), characteristic(s), propertie(s), method/process steps or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

Additionally, the section headings herein are provided for consistency with the suggestions under 37 CFR 1.77 or otherwise to provide organizational cues. These headings shall not limit or characterize the invention(s) set out in any claims that may issue from this disclosure. Specifically and by way of example, although the headings refer to a “Field of Invention,” such claims should not be limited by the language under this heading to describe the so-called technical field. Further, a description of technology in the “Background of the Invention” section is not to be construed as an admission that technology is prior art to any invention(s) in this disclosure. Neither is the “Summary” to be considered a characterization of the invention(s) set forth in issued claims. Furthermore, any reference in this disclosure to “invention” in the singular should not be used to argue that there is only a single point of novelty in this disclosure. Multiple inventions may be set forth according to the limitations of the multiple claims issuing from this disclosure, and such claims accordingly define the invention(s), and their equivalents, that are protected thereby. In all instances, the scope of such claims shall be considered on their own merits in light of this disclosure, but should not be constrained by the headings set forth herein.

For each of the claims, each dependent claim can depend both from the independent claim and from each of the prior dependent claims for each and every claim so long as the prior claim provides a proper antecedent basis for a claim term or element.

To aid the Patent Office, and any readers of any patent issued on this application in interpreting the claims appended hereto, applicants wish to note that they do not intend any of the appended claims to invoke paragraph 6 of 35 U.S.C. § 112, U.S.C. § 112 paragraph (f), or equivalent, as it exists on the date of filing hereof unless the words “means for” or “step for” are explicitly used in the particular claim.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### REFERENCES

[1] A.M. Schrand, L. Dai, J.J. Schlager, S.M. Hussain, Toxicity testing of nanomaterials., Advances in Experimental Medicine and Biology 745 (2012) 58-75.
[2] A. Helland, P. Wick, A. Koehler, K. Schmid, C. Som, Reviewing the environmental and human health knowledge base of carbon nanotubes, Environmental health perspectives 115(8) (2007) 1125-31.
[3] J.P. Ryman-Rasmussen, M.F. Cesta, A.R. Brody, J.K. Shipley-Phillips, J.I. Everitt, E.W. Tewksbury, O.R. Moss, B.A. Wong, D.E. Dodd, M.E. Andersen, J.C. Bonner, Inhaled carbon nanotubes reach the subpleural tissue in mice., Nature Nanotechnology 4(11) (2009) 747-51.
[4] M. Beck-Broichsitter, O.M. Merkel, T. Kissel, Controlled Pulmonary Drug and Gene Delivery Using Polymeric Nano-carriers, J Control Release 161(2) (2012) 214-224.
[5] J.J. Pippin, Animal Research in Medical Sciences: Seeking a Convergence of Science, Medicine, and Animal Law, South Texas Law Review 54 (2013) 469-511.
[6] S. De Minicis, E. Seki, H. Uchinami, J. Kluwe, Y. Zhang, D.A. Brenner, R.F. Schwabe, Gene Expression Profiles during Hepatic Stellate Cell Activation in Culture and in vivo, Gastroenterology 132(5) (2007) 1937-46.
[7] C.E. Barkauskas, M.-I. Chung, B. Fioret, X. Gao, H. Katsura, B.L.M. Hogan, Lung organoids: current uses and future promise, Development 144(6) (2017) 986-997.
[8] J. Kong, S. Wen, W. Cao, P. Yue, X. Xu, Y. Zhang, L. Luo, T. Chen, L. Li, F. Wang, Lung organoids, useful tools for investigating epithelial repair after lung injury, Stem Cell Research & Therapy 12(1) (2021) 1-13.
[9] D. Huh, B.D. Matthews, A. Mammoto, M. Montoya-Zavala, H.Y. Hsin, D.E. Ingber, Reconstituting Organ-level Lung Functions on a Chip, Science 328(5986) (2010) 1662-8.
[10] K. Ronaldson-Bouchard, G. Vunjak-Novakovic, Organs-on-a-Chip: A Fast Track for Engineered Human Tissues in Drug Development, Cell Stem Cell 22(3) (2018) 310-324.
[11] D. Huh, D.C. Leslie, B.D. Matthews, J.P. Fraser, S. Jurek, G.A. Hamilton, K.S. Thorneloe, M.A. McAlexander, D.E. Ingber, A human disease model of drug toxicity-induced pulmonary edema in a lungon-a-chip microdevice, Science Translational Medicine 4(159) (2012) 159ra147.
[12] K.H. Benam, R. Villenave, C. Lucchesi, A. Varone, C. Hubeau, H.H. Lee, S.E. Alves, M. Salmon, T.C. Ferrante, J.C. Weaver, A. Bahinski, G.A. Hamilton, D.E. Ingber, Small Airway-on-a-chip Enables Analysis of Human Lung Inflammation and Drug Responses in vitro, Nature Methods 13(2) (2016) 151-7.
[13] A. Sundarakrishnan, H. Zukas, J. Coburn, B.T. Bertini, Z.Y. Liu, I. Georgakoudi, L. Baugh, Q. Dasgupta, L.D. Black, D.L. Kaplan, Bioengineered in Vitro Tissue Model of Fibroblast Activation for Modeling Pulmonary Fibrosis, Acs Biomater Sci Eng 5(5) (2019) 2417-2429.
[14] J.C. Mejias, M.R. Nelson, O. Liseth, K. Roy, A 96-well Format Microvascularized Human Lung-on a-chip Platform for Microphysiological Modeling of Fibrotic Diseases, Lab Chip 20(19) (2020) 3601-3611.
[15] C. Wang, J. Yang, Mechanical forces: the missing link between idiopathic pulmonary fibrosis and lung cancer, European Journal of Cell Biology (2022) 151234.
[16] D. Pankova, Y. Jiang, M. Chatzifrangkeskou, I. Vendrell, J. Buzzelli, A. Ryan, C. Brown, E. O'Neill, RASSF 1A controls tissue stiffness and cancer stem-like cells in lung adenocarcinoma, The EMBO journal 38(13) (2019) e100532.
[17] K. Man, J. Liu, K.M. Phan, K. Wang, J.Y. Lee, X. Sun, M. Story, D. Saha, J. Liao, H. Sadat, Y. Yang, Dimensionality-Dependent Mechanical Stretch Regulation of Cell Behavior, ACS Applied Materials & Interfaces 14(15) (2022) 17081-17092.
[18] J.D. Stucki, N. Hobi, A. Galimov, A.O. Stucki, N. Schneider-Daum, C.M. Lehr, H. Huwer, M. Frick, M. Funke-Chambour, T. Geiser, O.T. Guenat, Medium throughput breathing human primary cell alveolus on-chip model, Sci Rep 8(1) (2018) 14359.
[19] O.Y.F. Henry, R. Villenave, M.J. Cronce, W.D. Leineweber, M.A. Benz, D.E. Ingber, Organs-on chips with Integrated Electrodes for Trans-epithelial Electrical Resistance (TEER) Measurements of Human Epithelial Barrier Function, Lab Chip 17(13) (2017) 2264-2271.
[20] R.R. Mercer, J.F. Scabilloni, A.F. Hubbs, L.A. Battelli, W. McKinney, S. Friend, M.G. Wolfarth, M. Andrew, V. Castranova, D.W. Porter, Distribution and Fibrotic Response Following Inhalation Exposure to Multi-walled Carbon Nanotubes, Particle and Fibre Toxicology 10 (2013) 33.
[21] J.S. Lwebuga-Mukasa, G. Thulin, J.A. Madri, C. Barrett, J.B. Warshaw, An acellular human amnionic membrane model for in vitro culture of type II pneumocytes: the role of the basement membrane in cell morphology and function, Journal of Cellular Physiology 121(1) (1984) 215-225.
[22] K. Brune, J. Frank, A. Schwingshackl, J. Finigan, V.K. Sidhaye, Pulmonary epithelial barrier function: some new players and mechanisms, American Journal of Physiology-Lung Cellular and Molecular Physiology 308(8) (2015) L731-L745.
[23] C.A. Goncalves, M.H. Figueiredo, V.A. Bairos, Three-dimensional organization of the elastic fibres in the rat lung, Anat Rec 243(1) (1995) 63-70.
[24] R.R. Mercer, A.F. Hubbs, J.F. Scabilloni, L. Wang, L.A. Battelli, S. Friend, V. Castranova, D.W. Porter, Pulmonary fibrotic response to aspiration of multi-walled carbon nanotubes., Particle and Fibre Toxicology 8 (2011) 21.

[25] G. Raghu, Y.Y. Chen, V. Rusch, P.S. Rabinovitch, Differential proliferation of fibroblasts cultured from normal and fibrotic human lungs, The American Review of Respiratory Disease 138(3) (1988) 703-8.
[26] D.V. Pechkovsky, A. Prasse, F. Kollert, K.M. Engel, J. Dentler, W. Luttmann, K. Friedrich, J. Muller-Quernheim, G. Zissel, Alternatively activated alveolar macrophages in pulmonary fibrosis-mediator production and intracellular signal transduction, Clin Immunol 137(1) (2010) 89-101.
[27] K. Wang, L. Shi, W. Linthicum, K. Man, X. He, Q. Wen, L.W. Rojanasakul, Y. Rojanasakul, Y. Yang, Substrate Stiffness-Dependent Carbon Nanotube-Induced Lung Fibrogenesis, Nano Letters 19(8) (2019) 5443-5451.
[28] E. Tzima, M. Irani-Tehrani, W.B. Kiosses, E. Dejana, D.A. Schultz, B. Engelhardt, G. Cao, H. DeLisser, M.A. Schwartz, A mechanosensory complex that mediates the endothelial cell response to fluid shear stress, Nature (London, United Kingdom) 437(7057) (2005) 426-431.
[29] C.P. Ng, M.A. Swartz, Fibroblast alignment under interstitial fluid flow using a novel 3-D tissue culture model, American Journal of Physiology-Heart and Circulatory Physiology 284(5) (2003) H1771-H1777.
[30] T. Mammoto, D.E. Ingber, Mechanical control of tissue and organ development, Development 137(9) (2010) 1407-1420.
[31] A. Tidu, D. Ghoubay-Benallaoua, B. Lynch, B. Haye, C. Illoul, J.M. Allain, V.M. Borderie, G. Mosser, Development of human corneal epithelium on organized fibrillated transparent collagen matrices synthesized at high concentration, Acta Biomaterialia 22 (2015) 50-58.
[32] S.J. Lai-Fook, R.E. Hyatt, Effects of Age on Elastic Moduli of Human Lungs, Journal of Applied Physiology 89(1) (2000) 163-8.
[33] D. Navajas, J. Alcaraz, R. Peslin, J. Roca, R. Farre, valuation of a Method for Assessing Respiratory Mechanics during Noninvasive Ventilation, European Respiratory Journal 16(4) (2000) 704-9.
[34] F. Liu, J.D. Mih, B.S. Shea, A.T. Kho, A.S. Sharif, A.M. Tager, D.J. Tschumperlin, Feedback amplification of fibrosis through matrix stiffening and COX-2 suppression, Journal of Cell Biology 190(4) (2010) 693-706.
[35] S. Wang, J.M. Tarbell, Effect of Fluid Flow on Smooth Muscle Cells in a 3-dimensional Collagen Gel Model Arteriosclerosis, Thrombosis, and Vascular Biology 20(10) (2000) 2220-5.
[36] C.P. Ng, B. Hinz, M.A. Swartz, Interstitial fluid flow induces myofibroblast differentiation and collagen alignment in vitro, Journal of cell science 118(20) (2005) 4731-4739.
[37] D. Wang, J. Tarbell, Modeling interstitial flow in an artery wall allows estimation of wall shear stress on smooth muscle cells, (1995).
[38] S.R. Chary, R.K. Jain, Direct measurement of interstitial convection and diffusion of albumin in normal and neoplastic tissues by fluorescence photobleaching, Proceedings of the National Academy of Sciences 86(14) (1989) 5385-5389.
[39] K.G. Birukov, J.R. Jacobson, A.A. Flores, S.Q. Ye, A.A. Birukova, A.D. Verin, J.G. Garcia, Magnitude-dependent Regulation of Pulmonary Endothelial Cell Barrier Function by Cyclic Stretch, The American Journal of Physiology-Lung Cellular and Molecular Physiology 285(4) (2003) L785-97.
[40] Y. Yang, K. Kulangara, J. Sia, L. Wang, K.W. Leong, Engineering of a microfluidic cell culture platform embedded with nanoscale features, Lab on a Chip 11(9) (2011) 1638-1646.
[41] T.S. Frost, L. Jiang, R.M. Lynch, Y. Zohar, Permeability of Epithelial/Endothelial Barriers in Transwells and Microfluidic Bilayer Devices, Micromachines 10(8) (2019) 533.
[42] Z. Liu, H. Wu, K. Jiang, Y. Wang, W. Zhang, Q. Chu, J. Li, H. Huang, T. Cai, H. Ji, MAPK-mediated YAP activation controls mechanical-tension-induced pulmonary alveolar regeneration, Cell reports 16(7) (2016) 1810-1819.
[43] X. Yang, K. Li, X. Zhang, C. Liu, B. Guo, W. Wen, X. Gao, Nanofiber membrane supported lung-on a-chip microdevice for anti-cancer drug testing, Lab on a Chip 18(3) (2018) 486-495.
[44] N. Higuita-Castro, M.T. Nelson, V. Shukla, P.A. Agudelo-Garcia, W. Zhang, S.M. Duarte-Sanmiguel, J.A. Englert, J.J. Lannutti, D.J. Hansford, S.N. Ghadiali, Using a novel microfabricated model of the alveolar-capillary barrier to investigate the effect of matrix structure on atelectrauma, Scientific Reports 7(1) (2017) 1-13.
[45] C.R.I. Lam, H.K. Wong, S. Nai, C.K. Chua, N.S. Tan, L.P. Tan, A 3D biomimetic model of tissue stiffness interface for cancer drug testing, Molecular pharmaceutics 11(7) (2014) 2016-2021.
[46] N. Kirschner, R. Rosenthal, M. Furuse, I. Moll, M. Fromm, J.M. Brandner, Contribution of tight junction proteins to ion, macromolecule, and water barrier in keratinocytes, Journal of Investigative Dermatology 133(5) (2013) 1161-1169.
[47] K.A. Foster, C.G. Oster, M.M. Mayer, M.L. Avery, K.L. Audus, Characterization of the A549 cell line as a type II pulmonary epithelial cell model for drug metabolism, Experimental cell research 243(2) (1998) 359-366.
[48] T.S. Frost, L. Jiang, Y. Zohar, Pharmacokinetic analysis of epithelial/endothelial cell barriers in microfluidic bilayer devices with an air-liquid interface, Micromachines 11(5) (2020) 536.
[49] Y. Zhu, C.M. Sköld, X. Liu, H. Wang, T. Kohyama, F.-Q. Wen, R.F. Ertl, S.I. Rennard, Fibroblasts and monocyte macrophages contract and degrade three-dimensional collagen gels in extended co-culture, Respiratory research 2(5) (2001) 1-7.
[50] D. Eyrich, F. Brandl, B. Appel, H. Wiese, G. Maier, M. Wenzel, R. Staudenmaier, A. Goepferich, T. Blunk, Long-term Stable Fibrin Gels for Cartilage Engineering, Biomaterials 28(1) (2007) 55-65.
[51] E.D. Grassl, T.R. Oegema, R.T. Tranquillo, Fibrin as an alternative biopolymer to type-I collagen for the fabrication of a media equivalent, J Biomed Mater Res 60(4) (2002) 607-612.
[52] T.L. Tuan, A. Song, S. Chang, S. Younai, M.E. Nimni, In vitro fibroplasia: matrix contraction, cell growth, and collagen production of fibroblasts cultured in fibrin gels, Exp Cell Res 223(1) (1996) 127-34.
[53] A.J. Gray, J.E. Bishop, J.T. Reeves, R.P. Mecham, G.J. Laurent, Partially degraded fibrin(ogen) stimulates fibroblast proliferation in vitro, Am J Respir Cell Mol Biol 12(6) (1995) 684-90.
[54] V.K. Lai, S.P. Lake, C.R. Frey, R.T. Tranquillo, V.H. Barocas, Mechanical behavior of collagen-fibrin co-gels reflects transition from series to parallel interactions with increasing collagen content, J Biomech Eng 134(1) (2012) 011004.
[55] J.M. Sucre, C.S. Jetter, H. Loomans, J. Williams, E.J. Plosa, J.T. Benjamin, L.R. Young, J.A. Kropski, C.L. Calvi, S. Kook, Successful establishment of primary type II alveolar epithelium with 3D organotypic coculture, American journal of respiratory cell and molecular biology 59(2) (2018) 158-166.
[56] H. Fehrenbach, Alveolar epithelial type II cell: defender of the alveolus revisited, Respiratory research 2(1) (2001) 1-20.
[57] H. Duong, B. Wu, B. Tawil, Modulation of 3D fibrin matrix stiffness by intrinsic fibrinogen-thrombin compositions and by extrinsic cellular activity, Tissue engineering. Part A 15(7) (2009) 1865-76.
[58] L. Si, H. Bai, M. Rodas, W. Cao, C.Y. Oh, A. Jiang, R. Moller, D. Hoagland, K. Oishi, S. Horiuchi, S. Uhl, D. Blanco-Melo, R.A. Albrecht, W.-C. Liu, T. Jordan, B.E. Nilsson-Payant, I. Golynker, J. Frere, J. Logue, R. Haupt, M. McGrath, S. Weston, T. Zhang, R. Plebani, M. Soong, A. Nurani, S.M. Kim, D.Y. Zhu, K.H. Benam, G. Goyal, S.E. Gilpin, R. Prantil-Baun, S.P. Gygi, R.K. Powers, K.E. Carlson, M. Frieman, B.R. tenOever, D.E. Ingber, A Human-airway-on-a-chip for the Rapid Identification of Candidate Antiviral Therapeutics and Prophylactics, Nature Biomedical Engineering (2021).
[59] E.Y. Bissonnette, J.F. Lauzon-Joset, J.S. Debley, S.F. Ziegler, Cross-Talk Between Alveolar Macrophages and Lung Epithelial Cells is Essential to Maintain Lung Homeostasis, Frontiers in Immunology 11 (2020).
[60] G. Oberdörster, E. Oberdörster, J. Oberdörster, Nanotoxicology: an emerging discipline evolving from studies of ultrafine particles, Environmental health perspectives 113(7) (2005) 823-839.
[61] S. Hussain, S. Sangtian, S.M. Anderson, R.J. Snyder, J.D. Marshburn, A.B. Rice, J.C. Bonner, S. Garantziotis, Inflammasome activation in airway epithelial cells after multi-walled carbon nanotube exposure mediates a profibrotic response in lung fibroblasts, Particle and fibre toxicology 11(1) (2014) 1-16
[62] S. Luanpitpong, L. Wang, A. Manke, K.H. Martin, A.G. Ammer, V. Castranova, Y. Yang, Y. Rojansakul, Induction of stemlike cells with fibrogenic properties by carbon nanotubes and its role in fibrogenesis, Nano Letters 14(6) (2014) 3110-6.
[63] K. Wang, X. He, W. Linthicum, R. Mezan, L. Wang, Y. Rojanasakul, Q. Wen, Y. Yang, Carbon Nanotubes Induced Fibrogenesis on Nanostructured Substrates, Environ Sci Nano 4(3) (2017) 689-699.
[64] D.W. Porter, A.F. Hubbs, R.R. Mercer, N. Wu, M.G. Wolfarth, K. Sriram, S. Leonard, L. Battelli, D. Schwegler-Berry, S. Friend, Mouse pulmonary dose-and time course-responses induced by exposure to multi-walled carbon nanotubes, Toxicology 269(2-3) (2010) 136-147.
[65] P. Ruenraroengsak, S. Chen, S. Hu, J. Melbourne, S. Sweeney, A.J. Thorley, J.N. Skepper, M.S.P. Shaffer, T.D. Tetley, A.E. Porter, Translocation of functionalized multi-walled carbon nanotubes across human pulmonary alveolar epithelium: dominant role of epithelial type 1 cells, ACS nano 10(5) (2016) 5070-5085.
[66] Y. SY, R. RR, Chapter 3, Methods for Measuring Permeability, Regulation of Endothelial Barrier Function, Morgan & Claypool Life Sciences 2010.

What is claimed is:

1. A 3D microfluidic device mimicking an anatomy and physiology of a human lung alveolar interstitium comprising:
a housing defining a cavity, at least one inlet and one outlet, wherein the inlet and outlet are in fluid communication with an inlet chamber and an outlet chamber;
an interstitium chamber positioned in the cavity that comprises an electrospun nanofibrous membrane that supports growth of lung epithelial cells and fibroblasts, wherein the electrospun nanofibrous membrane is in fluid communication with the inlet chamber and an outlet chamber to provide a growth media to the lung epithelial cells and fibroblasts;
a pneumatic chamber separated from the interstitium chamber by a water-impermeable membrane and a source of air in fluid communication with the pneumatic chamber; and
an air chamber in fluid communication a second side of the with electrospun nanofibrous membrane of the interstitium chamber, wherein the air chamber is opposite the pneumatic chamber;
wherein integration of inputs and outputs mimics the anatomy and physiology of the human lung alveolar interstitium.

2. The microfluidic device of claim 1, wherein an inlet aperture and an outlet aperture allow for injection of at least one of fluid, gas and solid material within and through the device.

3. The microfluidic device of claim 1, wherein the device is configured to be filled with cellular components on the electrospun nanofibrous membrane.

4. The microfluidic device of claim 1, further comprising at least one of:
one or more seals that separate the interstitium chamber, pneumatic chamber, and air chamber;
a pump to provide pneumatic pressure in the pneumatic chamber; or
the device comprises a material selected from a group consisting of glass, silicon, polysiloxane, polydimethylsiloxane, and optically transparent polymers.

5. The microfluidic device of claim 1, wherein the electrospun nanofibrous membrane has at least one of: physiological interstitial matrix stiffness or physiological 3D breathing mechanical stretch.

6. The microfluidic device of claim 1, wherein the interstitium chamber further comprises at least one of:
a collagen I-fibrin blend gel, or
one or more lung cell lines, lung primary cell cultures, alveolar epithelial cells, fibroblasts, immune cells or combinations thereof.

7. A lab-on-a-chip comprising a 3D microfluidic device mimicking an anatomy and physiology of a human lung alveolar interstitium comprising:
a housing defining a cavity, at least one inlet and one outlet, wherein the inlet and outlet are in fluid communication with an inlet chamber and an outlet chamber;
an interstitium chamber positioned in the cavity that comprises an electrospun nanofibrous membrane that supports growth of lung epithelial cells and fibroblasts, wherein the electrospun nanofibrous membrane is in fluid communication with the inlet chamber and an outlet chamber to provide a growth media to the lung epithelial cells and fibroblasts;
a pneumatic chamber separated from the interstitium chamber by a water-impermeable membrane and a source of air in fluid communication with the pneumatic chamber; and
an air chamber in fluid communication a second side of the with electrospun nanofibrous membrane of the interstitium chamber, wherein the air chamber is opposite the pneumatic chamber;
wherein integration of inputs and outputs mimics the anatomy and physiology of the human lung alveolar interstitium;
wherein an inlet aperture and an outlet aperture allow for injection of at least one of fluid, gas and solid material within and through the device.

8. The lab-on-a-chip of claim 7, wherein the device is configured to be filled with cellular components on the electrospun nanofibrous membrane.

9. The lab-on-a-chip of claim 7, further comprising at least one of:
one or more seals that separate the interstitium chamber, pneumatic chamber, and air chamber;
a pump to provide pneumatic pressure in the pneumatic chamber; or
the device comprises a material selected from a group consisting of glass, silicon, polysiloxane, polydimethylsiloxane, and optically transparent polymers.

10. The lab-on-a-chip of claim 7, wherein the electrospun nanofibrous membrane has at least one of: physiological interstitial matrix stiffness or physiological 3D breathing mechanical stretch.

11. The lab-on-a-chip of claim 7, wherein the interstitium chamber further comprises at least one of;
a collagen I-fibrin blend gel; or
the interstitium chamber comprises one or more lung cell lines, lung primary cell cultures, alveolar epithelial cells, fibroblasts, or combinations thereof.

12. A kit comprising a lab-on-a-chip comprising:
a 3D microfluidic device mimicking an anatomy and physiology of a human lung alveolar interstitium comprising:
a housing defining a cavity, at least one inlet and one outlet, wherein the inlet and outlet are in fluid communication with an inlet chamber and an outlet chamber;
an interstitium chamber positioned in the cavity that comprises an electrospun nanofibrous membrane that supports growth of lung epithelial cells and fibroblasts, wherein the electrospun nanofibrous membrane is in fluid communication with the inlet chamber and an outlet chamber to provide a growth media to the lung epithelial cells and fibroblasts;
a pneumatic chamber separated from the interstitium chamber by a water-impermeable membrane and a source of air in fluid communication with the pneumatic chamber; and
an air chamber in fluid communication a second side of the with electrospun nanofibrous membrane of the interstitium chamber, wherein the air chamber is opposite the pneumatic chamber;
wherein integration of inputs and outputs mimics the anatomy and physiology of the human lung alveolar interstitium.

13. A method mimicking an anatomy and physiology of a human lung alveolar interstitium with a lab-on-a-chip comprising a 3D microfluidic device comprising:
providing a housing defining a cavity, at least one inlet and one outlet, wherein the inlet and outlet are in fluid communication with an inlet chamber and an outlet chamber;
inserting lung epithelial cells and fibroblasts into an interstitium chamber positioned in the cavity that comprises an electrospun nanofibrous membrane that supports growth of the lung epithelial cells and fibroblasts, wherein the electrospun nanofibrous membrane is in fluid communication with the inlet chamber and an outlet chamber to provide a growth media to the lung epithelial cells and fibroblasts;
applying pneumatic pressure to the device into a pneumatic chamber separated from the interstitium chamber by a water-impermeable membrane and a source of air in fluid communication with the pneumatic chamber; and
providing an air chamber in fluid communication a second side of the with electrospun nanofibrous membrane of the interstitium chamber, wherein the air chamber is opposite the pneumatic chamber;
wherein integration of inputs and outputs mimics the anatomy and physiology of the human lung alveolar interstitium.

14. The method of claim 13, wherein the inlet aperture and the outlet aperture allow for injection of at least one of fluid, gas and solid material within and through the device.

15. The method of claim 13, wherein the device is configured to be filled with cellular components in the electrospun nanofibrous membrane.

16. The method of claim 13, further comprising at least one of:
one or more seals that separate the interstitium chamber, pneumatic chamber, and air chamber;
a pump to provide pneumatic pressure in the pneumatic chamber; or
the device comprises a material selected from a group consisting of glass, silicon, polysiloxane, polydimethylsiloxane, and optically transparent polymers.

17. The method of claim 13, wherein the electrospun nanofibrous membrane has at least one of: physiological interstitial matrix stiffness or physiological 3D breathing mechanical stretch.

18. The method of claim 13, wherein the interstitium chamber further comprises at least one of:
a collagen I-fibrin blend gel; or
one or more lung cell lines, lung primary cell cultures, alveolar epithelial cells, fibroblasts, or combinations thereof.

19. A high-throughput method of determining the effectiveness of a candidate drug that impacts lung alveoli, the method comprising:
providing a housing defining an array of test wells that each comprise a first and a second cavity, each of the cavities comprising at least one inlet and one outlet, wherein the inlet and outlet are in fluid communication with an inlet chamber and an outlet chamber;
inserting lung epithelial cells and fibroblasts into an interstitium chamber positioned in the housing that comprises an electrospun nanofibrous membrane that supports the growth of the lung epithelial cells and fibroblasts, wherein the electrospun nanofibrous membrane is in fluid communication with the at least one inlet chamber and an outlet chamber to provide a growth media to the lung epithelial cells and fibroblasts;
applying pneumatic pressure into a pneumatic chamber separated from the interstitium chamber by a water-impermeable membrane and a source of air in fluid communication with the pneumatic chamber; and
providing an air chamber in fluid communication a second side of the electrospun nanofibrous membrane of the interstitium chamber, wherein the air chamber is opposite the pneumatic chamber;
wherein the integration of inputs and outputs mimics the anatomy and physiology of the human lung alveolar interstitium;
administering a candidate drug to at least a first test well, and a placebo to at least a placebo test well; and
determining if the candidate drug modifies one or more parameters associated with lung function in the at least first test well when compared to the placebo test well over a course of treatment with the candidate drug.
